(19) United States
(12) Patent Application Publication
(10) Pub. No.: US 2012/0107921 A1
(43) Pub. Date: May 3, 2012

(54) MODEL BASED CONTROLS FOR USE WITH BIOREACTORS

(75) Inventors: Bryan Dennis Willson, Fort Collins, CO (US); Michael R. Buehner, Fort Collins, CO (US); Peter Michael Young, Fort Collins, CO (US); David Jacob Rausen, Fort Collins, CO (US); Guy Robert Babbitt, Fort Collins, CO (US); Rich Schoonover, Fort Collins, CO (US); Kristina Weyer-Geigel, Fort Collins, CO (US); David Eli Sherman, Fort Collins, CO (US)

(73) Assignees: COLORADO STATE UNIVERSITY RESEARCH FOUNDATION, Fort Collins, CO (US); SOLIX BIOFUELS, INC., Fort Collins, CO (US)

(21) Appl. No.: 13/001,306

(22) PCT Filed: Jun. 26, 2009
(86) PCT No.: PCT/US09/48976

§ 371 Date: Jul. 1, 2011

Related U.S. Application Data
(60) Provisional application No. 61/076,103, filed on Jun. 26, 2008.
(60) Provisional application No. 61/185,059, filed on Jun. 8, 2009.

(51) Int. Cl. C12M 1/36; C12M 1/38; A01G 1/00

(52) U.S. Cl. 435/286.5; 47/1.4; 435/286.1; 435/286.6

(57) ABSTRACT

Embodiments of the present invention include model-based controls to control photobioreactor operation and the growth of algae for use as a biofuels feedstock. In some embodiments, the model-based control can accounts for future conditions such as weather, product pricing, customer demands and/or other variables to operate the reactors in a manner that optimizes product revenues, minimizes costs or energy, maximizes photosynthetic or energy balance efficiency, and/or any combination of the aforementioned factors.

### CROSS-REFERENCE TO RELATED APPLICATIONS

This application claims the benefit of U.S. Provisional Application No. 61/076,103, filed on Jun. 26, 2008 and U.S. Provisional Application No. 61/185,059, filed on Jun. 8, 2009 both of which are hereby incorporated by reference in their entirety for all purposes.

### FIELD

Embodiments of the present invention relate generally to modeling and control methodologies, and more specifically to modeling and control systems for use with bioreactors.

### BACKGROUND

Producing biofuels, such as biodiesel, bioethanol, and/or biogasoline, from renewable energy sources provides numerous benefits. The increasing costs, increasing difficulty of extraction, and depletion of known fossil fuel reserves help to spur the development of such alternative fuel supplies. Efforts have been made to develop renewable energy fuels such as ethanol from corn grain or biodiesel from canola, rapeseed and other sources. The amount of biofuel that can be derived from food plant materials is often limited and the underlying increase in food commodity prices often negatively impacts food availability in developing countries and food prices in the developed world.

Efforts are underway to generate biofuels from non-food materials, such as cellulosic ethanol from wood pulp, corn stover or sugar cane bagasse. Algae and other photosynthetic microorganisms can provide feedstock for biofuel synthesis. Biofuel production from algae could permit productivities per unit of land area orders of magnitude higher than those of corn, rapeseed, canola, sugar cane, and other traditional crops.

### SUMMARY

Systems and methods are described for modeling and control of bioreactors. Various embodiments of the present invention include model based control strategies for optimal operation of photobioreactors. In some embodiments of the present invention, a method for controlling algae growth in a photobioreactor (e.g., a flat panel photobioreactor) is provided. Any known species of algae or photosynthetic microorganisms may be grown in the photobioreactor and utilize such control strategies according to embodiments of the present invention. Any known species of algae, cyanobacteria or photosynthetic microorganisms may be grown in a photobioreactor or Algal Growth System (AGS) or Algae Growth System (AGS). Microorganisms suitable for growth in some embodiments of the present invention include, but are not limited to, Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, lsochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, lsochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp., which may be grown either separately or as a combination of species.

Some embodiments of the present invention can sense one or more environmental conditions to which a flat panel photobioreactor is subjected. Using the environmental conditions, a calculation of future growth of algae within the flat panel photobioreactor can be made with an algal growth model. In accordance with some embodiments, the algal growth model can relate growth of the algae to the one or more environmental conditions and to one or more operation parameters affecting algal growth. A selection operation can select the one or more operation parameters based on the calculation and then adjust one or more actuators to achieve the one or more operation parameters.

Some embodiments of the present invention provide for a system for growing algae that includes a photobioreactor, a modeling unit, a control unit, and an actuator unit. The photobioreactor can be subject to one or more environmental conditions (e.g., light, temperature, algal culture density, and/or media pH) and have one or more operation parameters (e.g., carbon delivery rate to the photobioreactor, media flow rate, and/or harvesting rate) that can be adjusted to affect growth of algae in the media. The modeling unit can include an algal growth model relating growth and constituents of the algae in the media to the one or more environmental conditions and the one or more operation parameters. The control unit can be configured to access the modeling unit and determine the one or more operation parameters based on the algal growth model. In some embodiments, the control unit can generate a control signal indicating the one or more operation parameters. The control signal can be transferred to the actuator unit that is configured to receive the control signal and adjust the one or more operation parameters based on the control signal.

In some embodiments, the control signal is a first control signal and the system further includes a sensor and a feedback control unit. The sensor can be configured to detect a sensed condition of the one or more environmental conditions and generate a sensing signal indicating the sensed condition. The feedback control unit can be configured to receive the sensing signal, compare the sensed condition with a setpoint condition, and generate a second control signal based on the comparison. The second control signal is communicated to the actuator unit that can be designed to receive the second control signal and adjust the one or more operation parameters based on the second control signal.

A photobioreactor in some embodiments of the present invention can include a network of sensors, a model of the photobioreactor, a carbon supply unit, and a determination unit. The network of sensors can be configured to sense a set of conditions associated with the photobioreactor. The model of the photobioreactor can predict algae growth from the set of conditions and a set of input variables that include carbon supply rate. In some embodiments, the model of the photobioreactor can include multiple subsystem models such as, but not limited to, a photosynthesis subsystem, a light subsystem, and/or a water chemistry subsystem. The carbon supply unit can include an actuator to control the carbon supply rate into the photobioreactor. The determination unit can use the model of the photobioreactor to determine the set of input variables that will result in a desired algae growth. In some embodiments, the determination unit can adjust the actuator to set the carbon supply rate based on the determined set of input variables.

In accordance with some embodiments, an adaptive control method can be used to control a photobioreactor. A sensing operation can sense one or more environmental conditions to which the photobioreactor is subjected. A growth calculation can calculate the growth of algae within the flat panel photobioreactor using an algal growth model, that relates growth of the algae to the one or more environmental conditions and to one or more operation parameters affecting algal growth. A selecting operation can then select the one or more operation parameters based on the calculation and one or more actuators can be adjusted to achieve the one or more operation parameters. A measurement operation measures an actual growth of algae within the flat panel photobioreactor. Using these measurements, at least a portion of the algal growth model can be updated based on the measurement such that a calculated growth of algae according to the algal growth model more closely resembles the actual growth of algae.

Some embodiments of the present invention include a system for harvesting algae from a photobioreactor containing media. The system can include a modeling module configured to calculate future growth of algae within the photobioreactor with an algal growth model that relates growth of the algae to one or more environmental conditions associated with the photobioreactor. Some embodiments include a harvesting module configured to calculate a harvest time at which a future growth of algae equals a predetermined threshold growth of algae and to generate a harvest signal indicating the harvest time.

Various embodiments of the present invention include a system for model-based diagnostics for determining if a possible malfunction exists. These systems can include a photobioreactor, a sensor, a modeling module, and an error generation module. The photobioreactor can contain media for growing algae. The sensor can be configured to detect an operating condition (e.g., daily algae growth) associated with a photobioreactor and generate a sensed value associated with the operating condition. The modeling module can be configured to generate an expected value associated with the operating condition based on an algal growth model that relates growth of the algae in the photobioreactor to one or more environmental conditions and the operation condition. The error generation module can be configured to generate an error signal when a difference between the sensed value and the expected value exceeds a predetermined threshold. In some embodiments, the predetermined threshold can change with respect to time. The sensed value and/or the expected value can be operating condition trends over time in some embodiments. According to some embodiments error generation unit can generate one or more error indicators when the expected value exceeds the predetermined threshold.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the scope of the present invention. Accordingly, the drawings and detailed description are to be regarded as illustrative in nature and not restrictive.

### BRIEF DESCRIPTION OF THE DRAWINGS

Embodiments of the present invention will be described and explained through the use of the accompanying drawings in which:

FIG. 1 illustrates an example of a photobioreactor system with two simultaneous control loops, one for gas, one for liquid, which can use different, similar, or identical growth models for calculating feedforward terms in accordance with some embodiments of the present invention;

FIG. 2 illustrates an example of a photobioreactor with a gas control loop in accordance with one or more embodiments of the present invention;

FIG. 3 illustrates an example of a photobioreactor with a solid and/or liquid control loop in accordance with various embodiments of the present invention;

FIG. 4 illustrates a complete integrated system for controlling algae growth with a high level feedforward-plus-feedback control system that regulates the gas and/or liquid flow rates into and out of a photobioreactor in accordance with some embodiments of the present invention;

FIG. 5 illustrates various techniques for modeling algae in a photobioreactor in accordance with one or more embodiments of the present invention;

FIG. 6 illustrates a model that can be used in one or more components of the implementation of a control system in accordance with various embodiments of the present invention;

FIG. 7 shows a model of a photobioreactor as a set of three interacting subsystems in accordance with some embodiments of the present invention;

FIG. 8 is a block diagram illustrating the use of a feedforward controller plus a feedback controller to regulate pH via CO₂ addition to a photobioreactor in accordance with one or more embodiments of the present invention;

FIG. 9 is flowchart showing an exemplary set of operations for using a feedforward controller plus a feedback controller to regulate pH via CO₂ addition to a photobioreactor in accordance with various embodiments of the present invention;

FIG. 10 illustrates a system for controlling a photobioreactor with feedforward and feedback controllers using an observer corrected model in accordance with one or more embodiments of the present invention;

FIG. 11 is a block diagram showing an example of an observer corrected growth model that may be used as a feedforward pH controller in accordance with some embodiments of the present invention;

FIG. 12 is a block diagram illustrating an example of an implementation of a controller using feedforward control and feedback in accordance with some embodiments of the present invention;

FIG. 13 is a flowchart showing an example of a set of operation for the implementation of a controller using feedforward control and feedback in accordance with one or more embodiments of the present invention;

FIG. 14 is a block diagram showing an example implementation of a controller using feedforward control combined with feedback in accordance with one or more embodiments of the present invention;

FIG. 15 is a flowchart illustrating an example of a set of operations for the implementation of a controller using feedforward control combined with feedback in accordance with various embodiments of the present invention;

FIG. 16 is a block diagram with an example of a gas control system with static input parameters to a growth model in a feedforward component in accordance with various embodiments of the present invention;

FIG. 17 is a flowchart illustrating an example of a set of operations for a gas control system with static input parameters to a growth model in a feedforward component in accordance with some embodiments of the present invention;

FIG. 18 is a graph illustrating the equilibrium pH versus carbon dioxide concentration in sparge gas in accordance with various embodiments of the present invention;

FIG. 19 illustrates an example of an intermittent gas delivery scheme in accordance with some embodiments of the present invention;

FIG. 20 is a block diagram illustrating an example of a liquid control system with model-based feedforward components in accordance with one or more embodiments of the present invention;

FIG. 21 illustrates an example of a Labview implementation of a feedback controller with anti-windup with which some embodiments of the present invention may be utilized;

FIG. 22 is a block diagram illustrating an example of a predictive control system that uses a controller that predicts future events to compute control actions in accordance with some embodiments of the present invention;

FIG. 23 is a block diagram illustrating an example of a predictive control system in accordance with one or more embodiments of the present invention;

FIG. 24 is a flowchart with a set of exemplary operations that may be used to implement a predictive control strategy in accordance with various embodiments of the present invention;

FIG. 25 is a block diagram illustrating an example architecture for a predictive control system in accordance with one or more embodiments of the present invention;

FIG. 26 is a block diagram illustrating an example architecture for a predictive control system with predictive pH regulation using a PAR prediction along with the growth model and pH feedback in accordance with one or more embodiments of the present invention;

FIG. 27 illustrates a block diagram showing with an exemplary set of components for the implementation of a controller using open loop predictive pH regulation using a growth model in accordance with various embodiments of the present invention;

FIG. 28 is a block diagram illustrating an example architecture for an adaptive control system in accordance with one or more embodiments of the present invention;

FIG. 29 is a block diagram illustrating an example architecture for an adaptive learning control system in accordance with one or more embodiments of the present invention;

FIG. 30 is a flowchart illustrating an exemplary set of operations for the operation of an adaptive control system that may be used with various embodiments of the present invention;

FIG. 31 is a block diagram illustrating an exemplary set of components for implementing a controller with adaptive feedforward control along with feedback pH regulation with feedforward dead-time compensation in accordance with some embodiments of the present invention;

FIG. 32 is a block diagram illustrating an exemplary set of components for implementing a controller with adaptive feedforward control along with feedback pH regulation with Smith predictor dead-time compensation in accordance with some embodiments of the present invention;

FIG. 33 illustrates a block diagram showing a predictive control system that may be used with some embodiments of the present invention;

FIG. 34 illustrates an example of a fault detection based supervisory control system that may be use in one or more embodiments of the present invention;

FIG. 35 is a flowchart illustrating an exemplary set of operations that may be used for fault detection based supervisory control in accordance with various embodiments of the present invention; and

FIG. 36 illustrates an example of a computer system with which embodiments of the present invention may be utilized.

Similarly, some components and/or operations may be separated into different blocks or combined into a single block for the purposes of discussion of some of the embodiments of the present invention. Moreover, while the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Researchers are exploring growing algae as a feedstock for biodiesel. In many designs the algae is grown inside closed reactors comprised of glass, plastic, flexible film, composite materials, and/or other materials known to those of ordinary skill in the art. Examples of closed system bioreactors suitable for growth of algae and other microorganisms are described in U.S. patent application Ser. No. 11/871,728, filed Oct. 12, 2007, which is incorporated by reference herein in its entirety.

The growth of algae in a bioreactor depends on various factors and significantly increased performance can be obtained if the concentrations of many of the system variables can be controlled. For example, the amount of CO₂ added to the media may directly control the productivity of the system. Some systems control the amount of CO₂ added to the water to control the pH; however, these control systems are often simple and based only on closed loop feedback based on a pH reading or operate in a simple open loop manner. In such cases, many of the system variables are not controlled, which may result in less than optimal control.

Some embodiments of a bioreactor include a network of sensor, a model of the photobioreactor, a carbon supply unit, and a determination unit. The network of sensors can be configured to sense a set of conditions associated with the photobioreactor. The model can predict algae growth from the set of conditions and a set of input variable (e.g., carbon supply rate). In some embodiments, the carbon supply unit can be associated with an actuator to control the carbon supply rate into the photobioreactor. Then, the determination unit can use the model of the photobioreactor to determine the set of input variables that will result in a desired algae growth. Accordingly, the actuator may be configured to set the carbon supply rate based on the determined set of input variables can be adjusted.

Some embodiments of the present invention include a harvesting module that is configured to calculate a harvest time at which a future growth of algae equals a predetermined threshold growth of algae (e.g., between two to four grams per liter, up to five grams per liter or more). In one or more embodiments, the harvesting module can generate a harvest signal indicating a harvest time. In some embodiments, the harvesting signal is communicated to a harvesting unit that is configured to generate a harvesting command when the culture density exceeds a density point.

According to some embodiments of the present invention, photobioreactors may be used to grow algae or other photosynthetic microorganisms. Embodiments of the present invention result in desired, improved, and/or optimal biomass growth, oil production, energy consumption, efficiency of CO₂ utilization, and/or other important metrics of operation.

As used herein the terms “connected” or “coupled” and related terms are used in an operational sense and are not necessarily limited to a direct physical connection or coupling. Thus, for example, two devices may be coupled directly, or via one or more intermediary media, modules, or devices. As another example, devices may be coupled in such a way that information can be passed therebetween, while not sharing any physical connection with one another. Based on the disclosure provided herein, one of ordinary skill in the art will appreciate a variety of ways in which connection or coupling exists in accordance with the aforementioned definition.

As used herein, the phrase “in communication with” generally refers to direct and indirect communications for the exchange of information between two or more devices, modules, applications, systems, components, or the like. For example, two devices may be in communication with each other in such a way that information or access to the devices can be passed therebetween, while not sharing any direct physical connection.

As used herein the phrases “in some embodiments,” “according to some embodiments,” “in the embodiments shown,” “in other embodiments,” and the like generally mean the particular feature, structure, or characteristic following the phrase is included in at least one embodiment of the present invention, and may be included in more than one embodiment of the present invention. In addition, such phrases do not necessarily refer to the same embodiments or different embodiments.

The term “module” refers broadly to software, hardware, or firmware (or any combination thereof) components. Modules are typically functional components that can generate useful data or other output using specified input(s). A module may or may not be self-contained. An application program (also called an “application”) may include one or more modules, or a module can include one or more application programs.

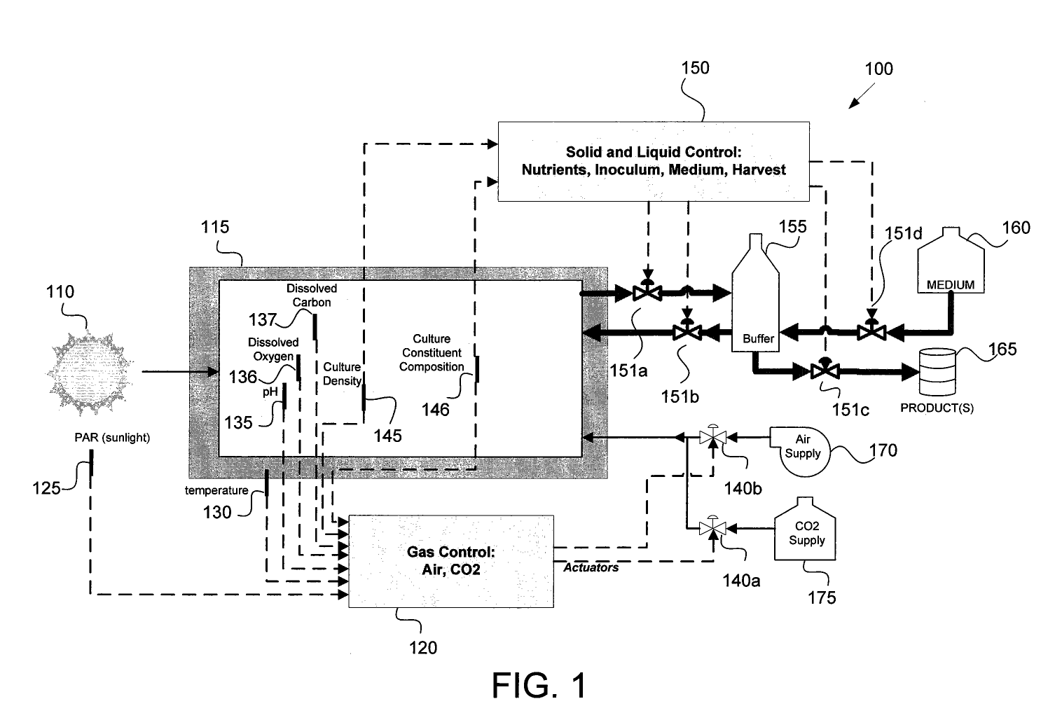

FIG. 1 illustrates an example of a photobioreactor system with two simultaneous control loops, one for gas, one for liquid, which can use different, similar, or identical growth models for calculating feedforward terms in accordance with some embodiments of the present invention. Integrated system 100 includes photobioreactor 115, various gas inlets and outlets, various liquid inlets and outlets, and two control systems. As illustrated in FIG. 1, gas control system 120 controls the flows of gases (e.g., air, CO₂) and liquid control system 150 controls the flows of liquids. Control systems 120 and 150 run either independently or in a coordinated manner. According to some embodiments, one or both of the control systems 120 and/or 150 include, or use information from, a model of the behavior of the photobioreactor that predicts its environmental conditions and/or operating parameters.

When light 110 (e.g., from the sun, lamps, etc) is projected on the photobioreactor 115, photosynthesis can occur, thereby causing growth of the algal culture. The algal consumes carbon dioxide from an air supply 170 whose flow is regulated by a valve or equivalent device and a carbon dioxide supply 175 whose flow is regulated by a valve, pump, or other flow-controlling device 140a. According to various embodiments, the flow-controlling devices can be operated automatically (e.g., through control of actuator) or manually. To achieve manual control of the valves, for example, the control system in some embodiments can provide instructions for manual processes (e.g., through a display device, lights, etc). As a result, oxygen is produced.

Liquids can be added to and/or removed from the photobioreactor via control valves, pumps, and/or manual processes. The gas control system 120 receives inputs of environmental conditions via sensors, hand measurements, or simulated environmental conditions generated by a model executing as part of or in conjunction with the control systems. The sensed, modeled, or hand-measured environmental conditions (or environmental inputs) may include a variety of parameters. Examples of environmental conditions include, but are not limited to, one or more of incident light such as photosynthetically active radiation 125, air and or water temperature 130, algal culture pH 135, dissolved oxygen 136, dissolved carbon 137, oxygen gas concentration, carbon gas concentration, dissolved carbon dioxide, algal culture density 145, and culture constituent levels 146.

According to various embodiments, the environmental conditions can be measured through sensors, received from one or more remote data suppliers (e.g., weather forecasts), estimated through chemical assays or other tests, predicted and/or estimated through models, measured by hand, and the like. In some cases, the sensors can be configured to detect a sensed condition of the one or more environmental conditions and generate a sensing signal indicating (or estimating) the sensed condition and/or environmental variable.

In some embodiments, levels of constituents that can be sensed, sampled, or modeled include culture lipids, culture lipid profile, beta carotene, protein, amino acids, glycerol, starches, hemicellulose, cellulose, waxes, chlorophylls, pigment molecules including carotenoids and xanthophylls, gamma linolenic acid, EPA (eicosapentaenoic acid 20:5n-3), DHA (docosahexaenoic acid 22:6n-3), ARA (arachidonic acid 20:4n-6), co-factors such as CoQ-10 or alpha-lipoic acid, molecules with antioxidant activity, and/or others. Control system 120 can use these sensed, sampled and/or modeled values to perform calculations that determine the desired operation parameters of flow rate and/or on and off times of the gas control valves 140a and 140b, according to embodiments of the present invention.

Liquid control system 150 can receive a measurement of the culture density 145 at some interval (e.g., every 15 seconds, every minute, every hour, etc). In addition, liquid control system 150 can receive an estimate of the culture density from the model. Using these measurements and/or estimates a determination can be made regarding the desired flow rates and timing of liquids that enter and leave the photobioreactor. These flow rates can include, but are not limited to, the flow of nutrient-containing medium from a medium source 160 to buffer tank 155. The embodiments illustrated in FIG. 1, use valve 151d or equivalent device to regulate the flow of medium and/or algal culture to buffer tank 155. The flow of the medium and/or algal culture from buffer tank 155 to photobioreactor 115 can be regulated by valve 151a or equivalent device. The flow of algal culture from photobioreactor 115 to the buffer tank 155 can be regulated by valve 151b or equivalent device. Similarly, the flow of products 165 from the buffer tank 155 can be regulated by valve 151c, pump, or flow-controlling equivalent device.

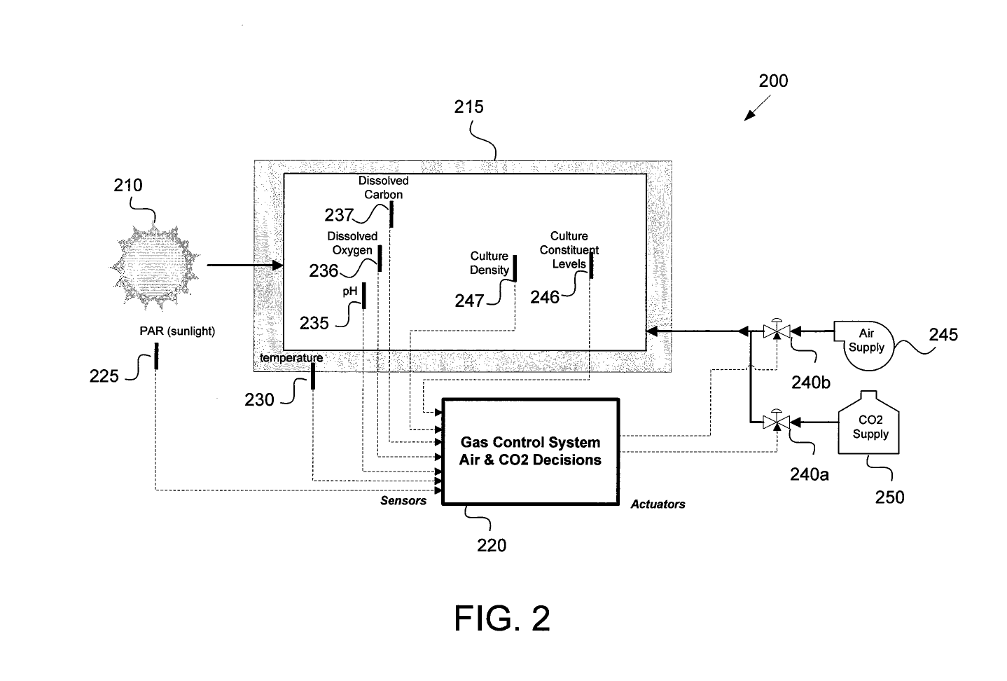

FIG. 2 illustrates an example of photobioreactor 215 with a gas control loop in accordance with one or more embodiments of the present invention. The gas control system 220 can operate as part of the integrated system of FIG. 1, or it can run independently to control the photobioreactor 215 without regard to the operation of any other control system, either manual or automated, including a control system that controls the flow of liquids in and out of the photobioreactor 215. In accordance with various embodiments, gas control system 220 can adjust one or more operation parameters via valves 240a and/or 240b in order to achieve one or more regulation objectives. Examples of regulation objectives include, but are not limited to, delivering air and/or carbon dioxide in order to achieve a desired pH and/or carbon concentration to achieve maximum algal growth and/or lipid production, maintaining dissolved oxygen in an acceptable range, maintaining adequate culture mixing to ensure culture health, optimal usage of available light and nutrients, maintaining flow of gases in order to minimize fouling of the photobioreactor 215, and/or others.

The performance objectives of gas control system 220 can include maximizing carbon dioxide utilization. Carbon dioxide utilization may be defined by the amount of carbon introduced to the system that is captured by photobioreactor 215. In some embodiments, gas control system 220 can be designed to minimize the energy usage and/or system cost required for the combined operation of the air supply system 245, the carbon dioxide supply system 250, the control valves 240a and 240b, and the control system 220 itself.

Similar to FIG. 1, in FIG. 2 examples of the environmental conditions that can be monitored, hand-sampled, predicted, received from external database, and/or modeled by the control system may include one or more of incident light such as photosynthetically active radiation 225, air and/or water temperature 230, algal culture pH 235, dissolved oxygen 236, dissolved carbon 237, algal culture density 247, and algal culture constituent levels 246 (e.g., constituent composition).

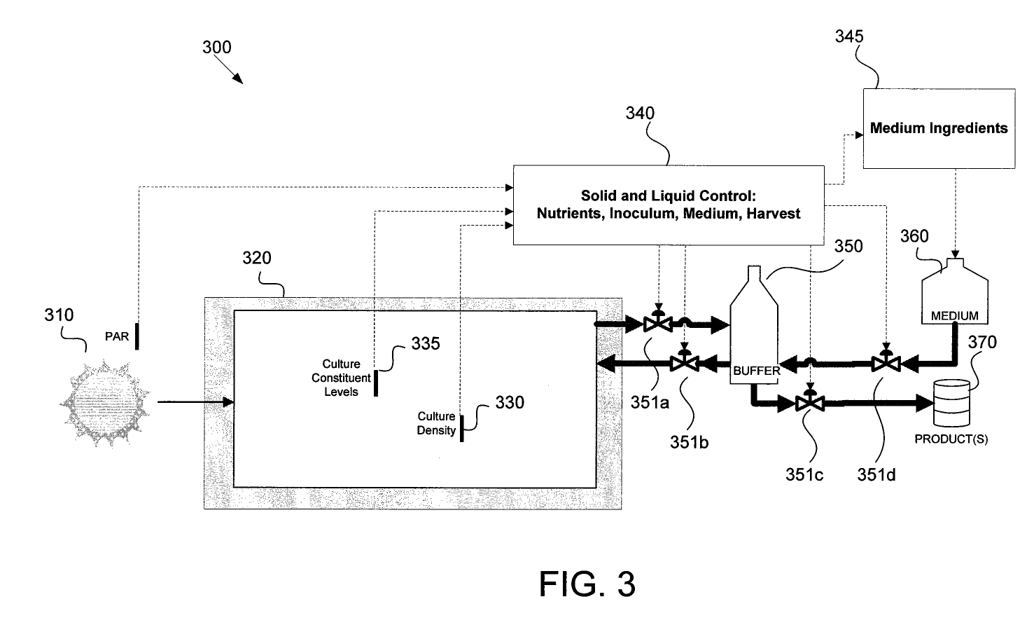

FIG. 3 illustrates an example of a photobioreactor 320 with a solid and/or liquid control loop in accordance with various embodiments of the present invention. The liquid control system 340 can operate as part of the integrated system of FIG. 1, or it can run independently to control the flows of liquids in and out of the photobioreactor 320. Control of the flows can be achieved by control system 340 directly controlling the actuators or by directing operation personnel (e.g., through a display device or light display) to set flow levels. As a result, control system 340 can run with or without regard to the operation of any other control system, either manual or automated, including a control system that controls the flow of gases in and out of the photobioreactor 320.

Liquid control system 340 monitors or estimates the values of culture conditions, including culture density 330 and culture constituent levels 335, in order to determine desired timing and rates of liquid flow into and out of the bioreactor 320. Flow rates are controlled in a manner similar to or identical to that of FIG. 1, via actuator units that are valves or equivalent devices 351a-d. In some embodiments, a solid control system may be a part of liquid control system 340 or may be independent of liquid control system 340. In some embodiments, a solid control system can generate a control signal to direct medium ingredients 345 to be added to medium 360.

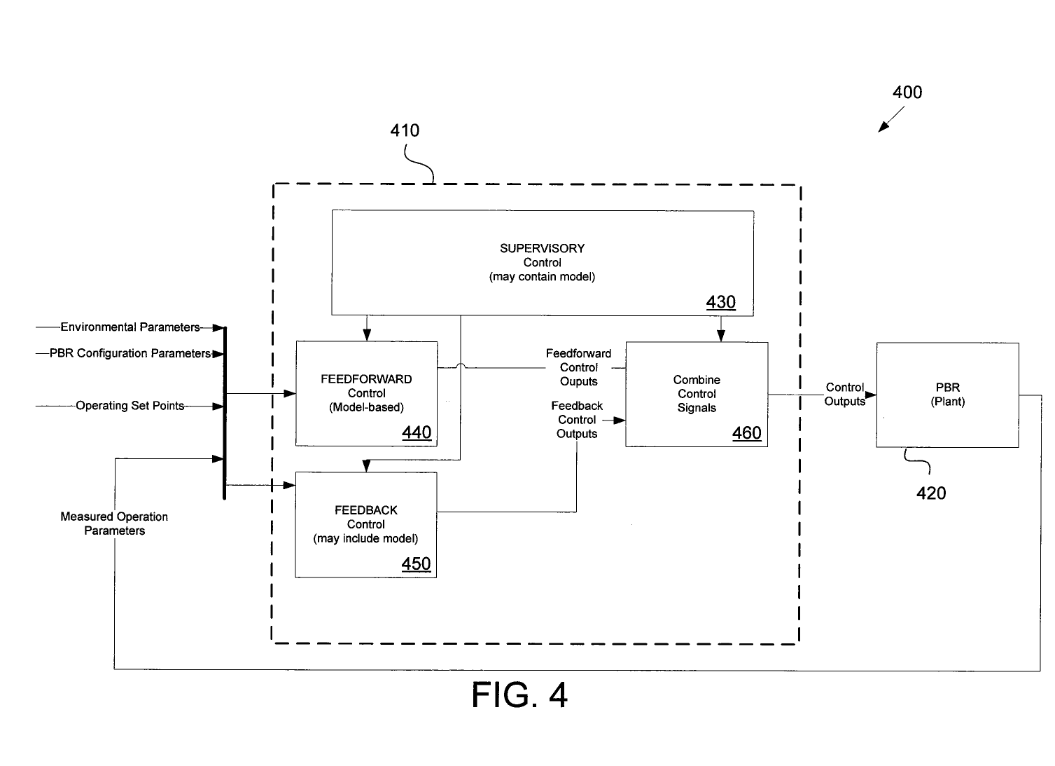

Various control systems and methodologies can be used to control algae growth within the photobioreactor. Examples include, but are not limited to, feedforward control, feedback control, model predictive control, adaptive control, and/or combinations of these and other control strategies. FIG. 4 illustrates a complete integrated system 400 for controlling algae growth with a high-level feedforward-plus-feedback control system 410 that regulates the gas and/or liquid flow rates into and out of a photobioreactor 420 in accordance with some embodiments of the present invention.

Supervisory control component 430 directs the behavior of a feedforward control module 440, feedback control module 450 and generation module 460 that combines control signals from the feedforward and feedback control modules in order to deliver control outputs to the photobioreactor plant 420. In accordance with various embodiments, supervisory control module 430 can perform calculations, provide enable/disable commands, setpoints, model calibration parameters, and/or operating mode commands to any of the three modules that receive inputs from supervisory control module 430.

Feedforward module 440 can use a set of actual measurements of environmental parameters, photobioreactor configuration parameters, operating setpoints, and/or photobioreactor plant measured operation parameters as inputs to, or in conjunction with, a simulation model that calculates the desired feedforward control outputs that will enable the operation parameters of the PBR plant to reach or approach desired values. According to some embodiments, the feedback control module 440 can calculate the difference between operating set points and actual measured operation parameters. In some embodiments, feedback control module 440 can also perform additional calculations as needed in order to determine on/off state or level of actuators that will enable the operation parameters of PBR plant 420 to reach or approach desired values.

Generation module combines the signals from feedforward control module 440 and feedback control module 450 in order to determine aggregated control signals for the control outputs, according to embodiments of the present invention. In some cases, the aggregation can be performed by summation or by using one signal or set of signals to enable or disable another signal or set of signals. In some embodiments, some or all of the functionality of any modules 430, 440, 450, and 460 can be combined into one module or performed by another module. For example, in some embodiments, modules 430, 440, 450, and 460 can be combined into a single integrated module in order to perform control in an optimal manner.

### Basic Control Algorithm

Described below are some examples of model-based controllers, according to embodiments of the present invention. According to some embodiments of the present invention, models of the organism and/or photobioreactor are used to calculate feedforward, or open loop, terms that control, completely or in part, one or more aspects of the photobioreactor system. For example, some embodiments of the present invention control the addition of carbon dioxide and/or nutrients.

According to other embodiments of the present invention, harvesting of the grown organism is performed with closed loop control to maintain continuous culture density or to cause the culture density to follow a command trajectory. A control algorithm is used to determine optimal cell density, according to embodiments of the present invention. According to such embodiments of the present invention, the control system continuously adjusts the rate at which the algae is harvested from the reactor to adjust the culture density to a desired density. This density may be measured directly using a turbidity meter (or similar method), inferred from other sensors, modeled, and/or measured “off line,” and the values entered back into the controller, according to embodiments of the present invention. A controller according to such embodiments maintains a constant culture density, or follows a culture density command trajectory. Such a command may be based on many factors including current reactor conditions, weather, product pricing, future weather and/or product pricing information.

Optimal cell density is a function of various factors and can vary from situation to situation. Rather that operating at one fixed density, some embodiments of the present invention determine an optimum density based on current conditions and predictions of future conditions such as weather, product demand, and/or product pricing. According to other embodiments, the cell density is controlled by controlling the harvest rate and/or dilution by the addition of media and/or inoculums. The inputs to the reactor are adjusted to match the current and future operating conditions (e.g., daily algal growth rate), according to embodiments of the present invention. Examples of control inputs include, but are not limited to, CO₂ addition, macronutrients such as nitrogen and phosphorus, micronutrients, sparging, harvesting, medium addition, reactor volume, reactor geometry, reactor configuration, and/or pumping.

According to some embodiments of the present invention, model-based control of a photobioreactor may be used for growth optimization and to maximize the values of all products. According to such embodiments, the model-based control may be used to improve growth rate, improve oil yield, minimize nutrient costs, minimize energy utilization, and/or minimize other operating costs. Models of the organism and/or the photobioreactor may be used to control the system in a feedback manner. The system may contain algae or other photosynthetic organisms, according to embodiments of the present invention.

The controls of the system may be used to optimize algae biomass production, lipid contents, and/or carbohydrates, according to embodiments of the present invention. Models of the organism and the photobioreactor may be used to determine how to control the photobioreactor system to maximize the combined values of all the products being harvested based on current reactor conditions, current weather and/or current product and co-product costs, according to embodiments of the present invention. In addition, different reactors may be controlled to achieve different results.

Model-based system diagnostics may also be used to determine if part, or all, of the photobioreactor system is operating incorrectly (i.e., some type of malfunction), according to embodiments of the present invention. According to some embodiments of the present invention, a biological model, a physical model, and/or an empirical model may be used to control a photobioreactor.

Model based control may be used to maximize the net values that can be gained for some or all products of a photobioreactor based on current or future estimates of product price, current or future constituent prices, upcoming weather, and/or other factors, according to embodiments of the present invention. According to such embodiments, predictions of future weather and product and co-product pricing may be used in conjunction with the system models to determine optimal operation of the photobioreactor to provide maximum value from all the products. This could include, for example, the control of harvesting rates, media addition, inoculum addition, nutrient addition, carbon dioxide addition, sparging rates, temperature, basin water levels, pressures in the system, pumping rates, and/or other means to mix the system.

According to some embodiments of the present invention, learning algorithms may be used to calibrate the photobioreactor system models and/or controllers, in which feedback may be employed to adapt or correct the system model and/or controllers to improve system performance. Such feedback systems may include various control formats, such as, for example, model-referencing adaptive control, neural nets, reinforcement learning, observers, and/or correction factors.

The following describes various ways in which a photobioreactor system may be controlled using forms of model-based control, adaptive learning, and/or prediction, reinforcement learning, according to embodiments of the present invention.

### 1 Model Based Control of an Algae Photobioreactor

According to some embodiments of the present invention, static and dynamic models are used to improve the productivity of bioreactors with an emphasis on growing microalgae in a photobioreactor (“PBR”). An algal growth model captures algal growth dynamics inside a closed reactor, which is used to dynamically compensate for changing conditions, according to embodiments of the present invention. While the model is independent of style of bioreactor, the specific examples presented here are for a flat panel photobioreactor, according to embodiments of the present invention. However, elongated tubular and airlift reactors may also be modeled by fitting different model parameters and using other simple dynamic models, for example first and second order, that match the physics of the bioreactor design, according to embodiments of the present invention. Embodiments of the present invention permit replacing sensors with models, maximizing performance (utilization and production), predicting future events and dynamically compensating ahead of time, and adapting to changing conditions.

This section outlines a model and its use for feedforward (“FF”) control in conjunction with a feedback (“FB”) controller, according to embodiments of the present invention. The topics for this section are:
The creation and/or use of a multi-domain model (e.g., physics, chemistry, and biological-based models)
FF/FB control of a bioreactor
Applications—growth, lipids, other byproducts, sensor replacement
Optimized Scheduling
Fault Detection

### 1.1 Multi Domain Modeling

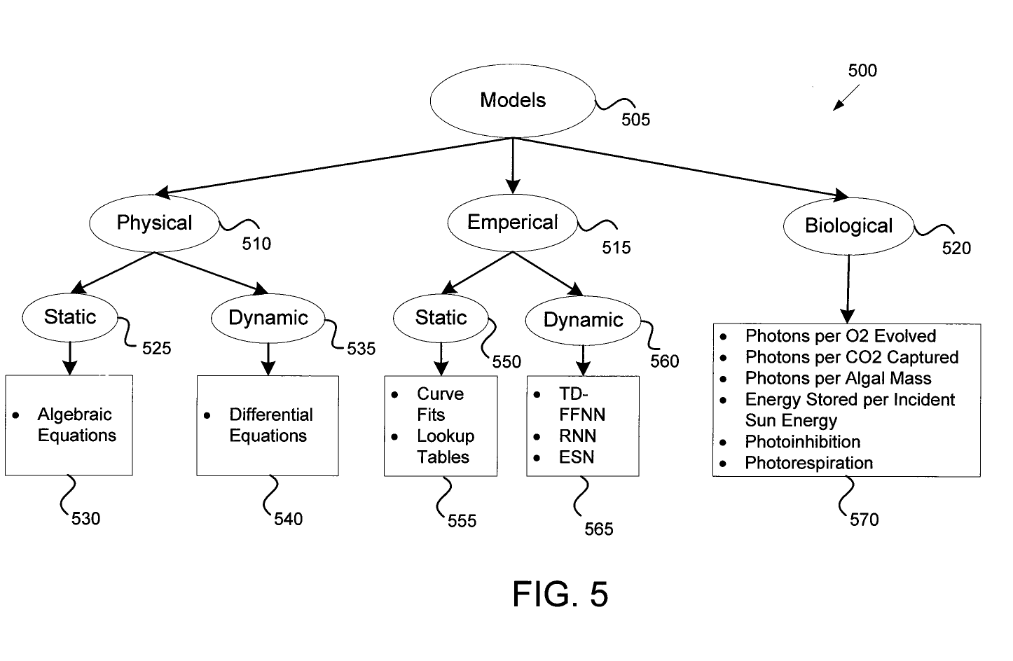

Various embodiments of the present invention use models to provide FF control and to synthesize FB controllers. FIG. 5 illustrates various techniques for modeling algae in a photobioreactor in accordance with one or more embodiments of the present invention. As illustrated in FIG. 5, there are three classifications of basic types of models 505 that can be used to model microalgae. Physical models 510, empirical models (e.g., fit from data) 515, and biologically based models 520 are three examples of models that can be used with embodiments of the present invention.

Physical models 510 include both static maps 525 (e.g., algebraic equations 530), dynamic models 535 (e.g., linear and nonlinear difference and differential equations 540), and/or the combination thereof. Empirical models 515 include both static models 550 (e.g., curve fits, algebraic expression, and/or lookup tables that employ inputs to generate the output 555) and dynamic models 560 (e.g., linear and nonlinear mappings that use one or more previous inputs or outputs along with the current input 565). In some embodiments, dynamic models 560 can use one or more memory elements while some static models 550 may be implemented without any stored values. Some examples include, but are not limited to, tap-delayed feedforward neural networks (TD-FFNN), recurrent neural networks (RNN), and echo state networks (ESN) 565. Biological models 520 can include modeling input output relations based on the known biological behavior (e.g., a known photosynthetic relationship according to which eight absorbed photons will produce one molecule of oxygen 570).

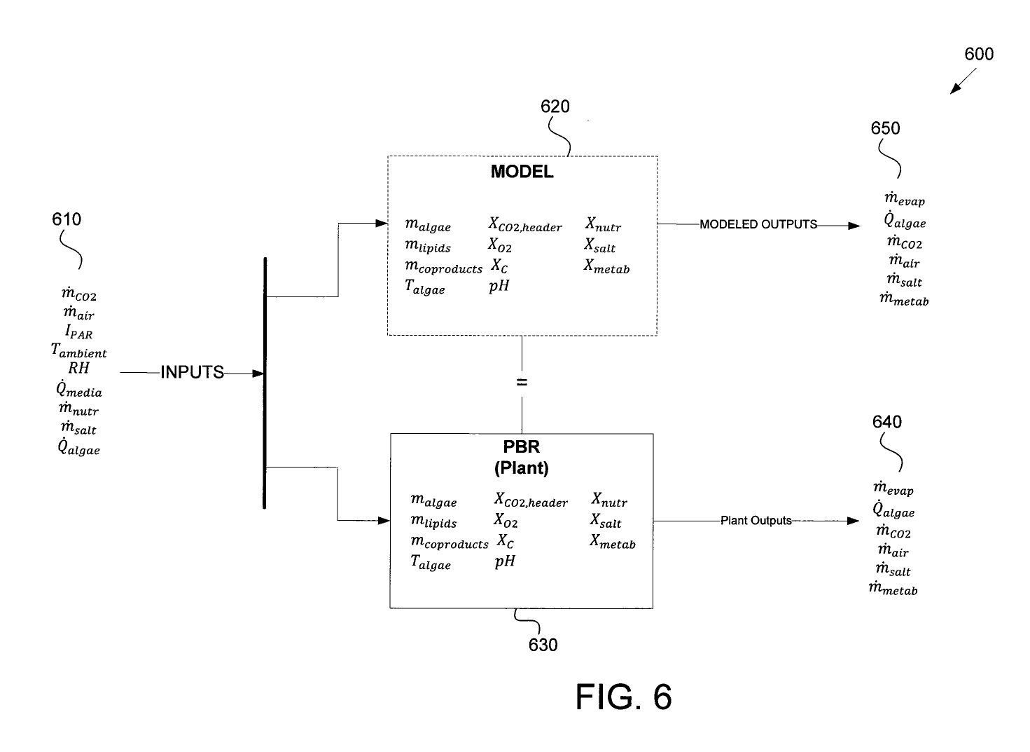

FIG. 6 illustrates a model 600 that can be used in one or more components of the implementation of a control system in accordance with various embodiments of the present invention. This model concept depicts that a model 620, executing or pre-executed on a computing device, partly or completely, replicates some of the behaviors in the actual PBR plant 630. The model can then be used in components (e.g., FEEDFORWARD and/or FEEDBACK) of the implementation of a control system for achieving some goal, such as gas control for supplying CO2 or liquid control for harvest.

Both the physical plant 630 and the plant model 620 receive environmental and operational parameters 610 as inputs. In various embodiments, the set of inputs to model 620 and to plant 630 may be the same or different. The physical parameters, including environmental conditions of plant 630, and the corresponding state variables of model 620 can be identical in some embodiments. Measurement of these state variables may be one method of validating, configuring, or calibrating the model. Plant 630 responds with actual outputs 640 (e.g., system flow rates of both liquids and gases). The model 620 can use inputs 610 to predict the outputs 650 of the plant 630.

A model used for FF control should accurately model the requirements of the algae, such as the nutrients and amount of CO₂ For the feedback control used in some embodiments of the present invention, the CO₂ requirement may be measured through a secondary pH measurement. CO₂ availability is closely related to pH. The rest of this section outlines a physical based model that can be used to control CO₂ delivery, according to embodiments of the present invention.

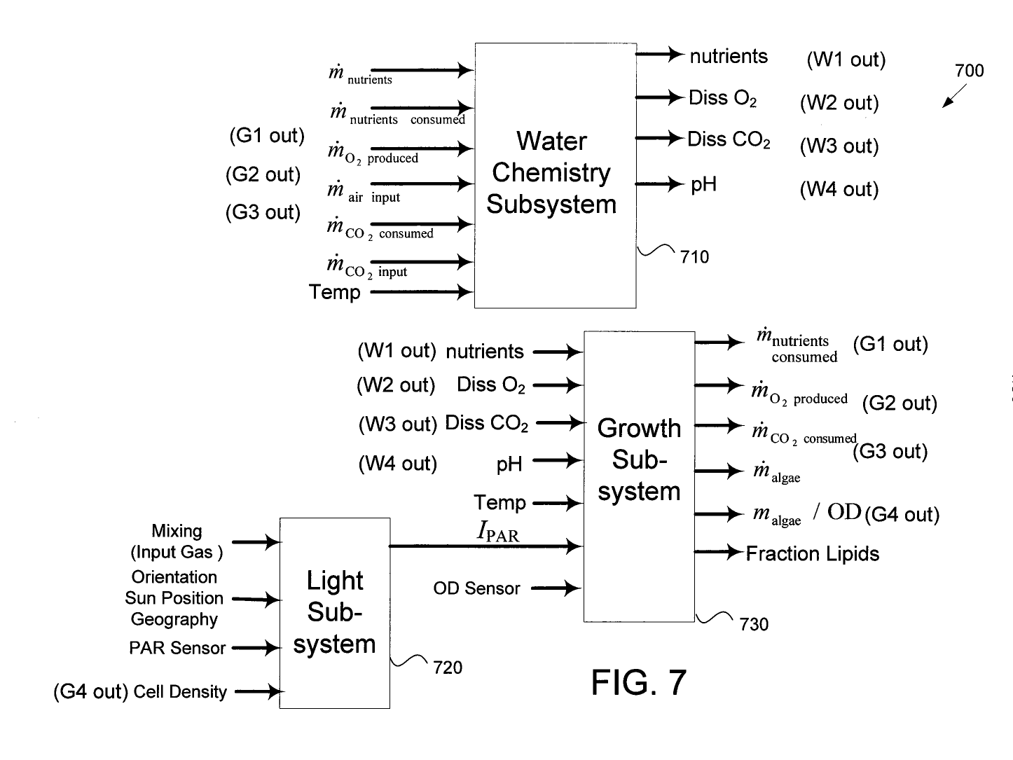

FIG. 7 shows a model 700 of the photobioreactor as a set of three interacting subsystems in accordance with some embodiments of the present invention. An overall PBR model 700, according to embodiments of the present invention, may be described as three main subsystems, namely the light subsystem 720, photosynthesis subsystem 730, and the water chemistry subsystem 710. The outputs of some subsystems can be inputs to the others, according to embodiments of the present invention. These outputs and their associated inputs are denoted by labels in parentheses.

All of the inputs to the model, except sunlight, may be commanded, according to embodiments of the present invention. This makes the control problem interesting, because sunlight is the input that drives photosynthesis, yet enters the system as an exogenous input. According to some embodiments of the present invention, the main goals of the model are to maximize growth (and hence CO₂ uptake) in the first stage and storage lipid accumulation in the second. The focus of this section will be on the growth model with a brief discussion of how the stress model relates to the growth model.

The control model for the growth phase includes trying to promote exponential growth during the sunlight, which means driving the system unstable, according to embodiments of the present invention. However, the system still requires nutrients and must remain within a safe pH and temperature. Therefore, this control problem may be solved with feedforward predictive control that predicts the amount of CO₂ required to maximize sun utilization in combination with a feedback controller that maintains safe operating conditions, according to embodiments of the present invention. In some embodiments, the modeling unit can produce a timing schedule for carbon delivery.

### 1.1.1 Incident Light Subsystem

The incident light subsystem determines the amount of light that will reach the microalgae, which is a function of intensity of sunlight reaching the reactor, sun position, amount of mixing, culture density, and/or PBR geometry, according to embodiments of the present invention. This section describes a model based on incident light. While mixing, culture density, and PBR geometry affect the amount of light received by the microalgae, these factors will be specific to a particular PBR setup. For the example reactors considered herein, these parameters are held constant. As a result, they will be grouped into a “sun utilization” constant and a critical density in the growth model, which is discussed in Section 1.1.2.

About 43% of the full spectrum of light is photosynthetically active radiation (“PAR”) which is the amount of light available for photosynthesis. Quantitatively, PAR is the light intensity in the 400 nm to 700 nm range. When the sun is out, the primary component of incident PAR is direct light, which will hit the bath water at a certain angle depending on the position of the sun. A portion of this light will reflect back off the water and some will enter the PBR bath. Not all of the light that enters the bath will be absorbed, and simple models can be used to capture enough information about the light in the reactor to provide a realistic growth model.

The total amount of PAR available for photosynthesis is a function of both the diffuse and direct light that enters the bath.

The amount of diffuse light entering the bath is a function of the sun position, weather (e.g., cloud cover, humidity, barometric pressure, and temperature), and surrounding reflective objects (e.g., buildings, structures, trees, and landscape).

The following derivation of reactor light is based on the information in another study. The amount of direct sunlight that enters the bath water is a function of the angle of incidence normal to the bath water. In turn, this angle is a function of the sun position, which depends upon the day of the year, time of day, and location (longitude and latitude). As the earth travels around the sun, the relative position of the sun in the sky changes with the seasons. This is captured by the sun declination, which is

$$δ = 23.4 sin ( 2 π \frac{360}{365} ( 284 + n ) ) \qquad ( 1 )$$

where 1≦n≦365 is the day of the year. The sun intensity is a function of solar time, where solar time is the local time adjusted so that the sun is the highest in the sky at solar noon. The conversion from local time to solar time is as follows:

$$\begin{matrix} B = \frac{360}{365} ( n - 1 ) & ( 2 ) \\ E = 0.000287 + 0.0072 cos ( 2 π B ) - 0.1225 sin ( 2 π B ) - 0.0558 cos ( 4 π B ) - 0.1562 sin ( 4 π B ) ) & ( 3 ) \\ D = \{ \begin{matrix}1 \text{:} n during daylight savings \\ 0 \text{:} n during standard time \end{matrix} & ( 4 ) \\ Δ t = \frac{( {L}_{st} - {L}_{loc} )}{15} + E - D & ( 5 ) \\ {t}_{solar} = {t}_{clock} + Δ t & ( 6 )\end{matrix}$$

In these equations, E is a correction in hours based on the day of the year (n). The variables Lₛₜ and L_{loc} are the standard and actual longitude values in degrees for the PBR location, and the flag variable D in equation 4 is equal to one when it is during daylight savings and zero otherwise. (The standard longitudes for the United States are 75° for the Eastern time zone, 90° for the Central time zone, 105° for the Mountain time zone, and 120° for the Pacific time zone).

The next parameter to calculate is the “hour angle,” which measures the number of degrees that the earth has traveled since solar noon. Because there are 360° of rotation in a 24 hour day, the earth travels 15 degrees every hour (hence the division by fifteen in eqn 5). The hour angle (in radians) is given by

ω=2π×[15(tₛₒₗₐᵣ−12)]. (7)

The angle of incidence (θ_{inc}) on a horizontal surface, such as the PBR bath, at a given latitude φₗₐₜ is

cos(θ_{inc})=cos(φₗₐₜ)cos(δ)cos(ω)+sin(φₗₐₜ)sin(δ) (8)

From Snell's Law, the angle of transmission into the water in which a photobioreactor is submerged, namely θ_{water}, is given by

$$\frac{{n}_{air}}{{n}_{water}} = \frac{sin ( {θ}_{air} )}{sin ( {θ}_{water} )} \qquad ( 9 )$$

where nₐᵢᵣ=1, n_{water}=1.333, and θₐᵢᵣ=θ_{inc} from equation 8. This is enough information to calculate water θ_{water}. To get the fraction of direct beam radiation that is transmitted through the water, two more variables are used, namely the perpendicular and parallel components of unpolarized radiation, which are given by

$$\begin{matrix}{r}_{⊥} = \frac{{sin ( {θ}_{water} - {θ}_{air} )}^{2 }}{{sin ( {θ}_{water} + {θ}_{air} )}^{2}} & ( 10 ) \\ {r}_{‖} = \frac{{tan ( {θ}_{water} - {θ}_{air} )}^{2}}{{tan ( {θ}_{water} + {θ}_{air} )}^{2 }} & ( 11 )\end{matrix}$$

From this, the reflectance is

$$\frac{{r}_{⊥} + {r}_{‖}}{2}$$

and the transmittance (or fraction of the light that enters the bath) is given by

$${η}_{bath} = 1 - \frac{{r}_{⊥} + {r}_{‖}}{2} \qquad ( 12 )$$

If PAR^{sun} is the amount of PAR from the sun, then the amount that will enter the PBR bath is

PAR_{bath}=η_{bath}PARₛᵤₙ (13)

The actual amount of PAR that the microalgae will use for photosynthesis is also a function of mixing and vertical flat panel geometry (i.e., panel thickness and orientation), according to embodiments of the present invention. Therefore, the amount of incident light available for algal photosynthesis will be

I_{PAR}=f₁(PAR_{bath},mixing,geometry). (14)

A simplified model of eqn 14 is

I_{PAR}=ηPBRPAR_{bath}, (15)

where η_{PBR} is the efficiency of the PBR for a given mixing and geometry. Currently, the term η_{PBR} is absorbed into the light utilization constant K_{PAR} in the next section. Therefore, I_{PAR}=PAR_{bath} is used for the growth model.

PAR may be measured in units of μmol light/m2/s; however, it is more convenient to convert PAR to units mol light/m2/h. The convenience comes from the fact that 8 moles of light should produce 1 mole of O₂ and that the growth rate is measured biomass produced per hour. This will become apparent in the next section. The conversion between the two PAR units is given by

I_{PAR(mol/m}_{₂}_{/h)}=0.0036I_{PAR(μmol/m}_{₂}_{/s)}. (16)

In addition to determining the amount of light the microalgae experience, the light subsystem also determines the culture density at which exponential growth becomes linear growth, according to embodiments of the present invention. This critical density, labeled m_{dense}, is a function of culture density, mixing, and PBR geometry. The details of this parameter and its meaning from a modeling perspective are discussed in the next section.

Photoinhibition is a phenomenon that occurs when the microalgae are exposed to an excess amount of light. Mixing is a method for minimizing the effects of photoinhibition and, in turn, utilizes more of the available sunlight inside a PBR. The rate at which mixing affects photoinhibition is a function of sun intensity on earth, cell density, and microalgae growth rate, according to embodiments of the present invention.

### 1.1.2 Photosynthesis Subsystem

The photosynthesis subsystem models the growth dynamics of the microalgae as it utilizes photons from the sun, CO₂, and nutrients to produce O₂ and more microalgae, according to embodiments of the present invention. The rate at which microalgae grow depends on their ability to utilize the incident light and on the availability of nutrients. Assuming there are ample nutrients available, the microalgae growth is primarily a function of input light. When there is an absence of light the microalgae respire (e.g. they utilize O₂ and stored carbon as an energy source), which releases CO₂ and results in a loss of biomass. In the presence of light, the microalgae both evolve O₂ as they assimilate carbon and respire O₂ as they consume stored carbon; however, the growth from carbon assimilation will often dominate the metabolic process. In some embodiments, the enzyme Rubisco can utilize both CO₂ and O₂ as substrates inside the microalgae.

When the culture is sparse, there are an excess number of light photons that are not being utilized. During this stage, the microalgae will grow exponentially, since the produced algal mass will not be limited by photons. At some point, the algal density will become great enough that all of the incident light will be utilized. At densities greater than this, the microalgae growth rate will be linear. As the density continues to grow, a smaller fraction of the microalgae will be able to receive the light required for photosynthesis and respiration will be the dominant metabolic activity. As this happens, the total microalgae growth in the PBR will cease and eventually begin to decay. In the model, this feature can be captured by saturating the density in the growth term. When the density gets above a critical density, labeled m_{dense}, the amount of growth resulting from photosynthesis becomes linear while the density lost due to respiration remains exponential. These effects are described by the following nonlinear differential equation:

$$\begin{matrix}{\dot{m}}_{algae} = P {\overline{m}}_{algae} - {Rm}_{algae} - {u}_{D} \\ where & ( 17 ) \\ P = {K}_{PAR} {I}_{PAR} \\ {\overline{m}}_{algae} = min ( {m}_{algae} , {m}_{dense} ) \\ {m}_{dense} = {f}_{2} ( {m}_{algae} , mixing , geometry ) & ( 18 )\end{matrix}$$

The state variable m_{algae} is the amount of microalgae inside the PBR (in units g/L) and its derivative, namely \dot{m}_{algae} (in units g/L/h), is the growth rate of microalgae inside the PBR. The productivity parameter P (in units 1/h) is the specific growth rate at a given sun intensity. The term K_{PAR} (in units m²/mol light) is the sun utilization constant that converts incident light, namely I_{PAR}, into microalgae growth rate. The variable R (in units 1/h) is the rate of biomass loss caused by respiration in the dark, u_{D} is the microalgae culture dilution rate from media replacement, and V_{PBR(L)} is the volume of the reactor. The last term is used in cases where the reactor is run in a continuous mode (i.e., when microalgae is continuously harvested and replaced with fresh media), according to embodiments of the present invention.

As microalgae grow, they consume carbon, which they get from CO₂ and other nutrients from their surroundings and release O₂. In general, microalgae biomass is 50% carbon by dry weight. A mole of CO₂ has a mass of 44 grams and 12 of these grams come from carbon. Based on these premises, the expression that 1 gram of microalgae can fix 1.83 grams of CO₂ may be derived as follows

$$\frac{44 {g}_{{CO}_{2}} / mol}{12 {g}_{C }} \frac{0.5 {g}_{C}}{{g}_{algae}} = \frac{11}{6} \frac{{g}_{{CO}_{2}}}{{g}_{algae}} \approx 1.83 \frac{{g}_{{CO}_{2}}}{{g}_{algae}} \qquad ( 19 )$$

A simplified equation for photosynthesis is given by

12H₂O+6CO₂+light→C₆H₁₂O₆+6O₂+6H₂O (20)

This equation establishes that for every gram of CO² consumed, there is a gram of O₂ produced. However, this is not the case because the O₂ molecules come from splitting water. Therefore, there is not a one-to-one correspondence of O₂ molecules produced to CO₂ molecules fixed. The excess energy that is not used to fix CO₂ is used for other metabolic processes (e.g., fixing nutrients from the surrounding media). This is often echoed in the literature by the fact that it takes eight photons of light to produce one O₂ molecule, but that it takes eight to sixteen photons of light to assimilate a CO₂ molecule.

Assuming that 10 photons of light are required to fix one CO² molecule, the amount of O₂ produced will be

$$\frac{32 {g}_{{O}_{2}} / {mol}_{{O}_{2}}}{44 {g}_{{CO}_{2}} / {mol}_{{CO}_{2}}} \frac{8 {mol}_{{O}_{2}}}{10 {mol}_{{CO}_{2}}} \frac{11 {g}_{{CO}_{2}}}{6 {g}_{algae}} = \frac{16 {g}_{{O}_{2}}}{15 {g}_{algae}} \approx 1.07 \frac{{g}_{{CO}_{2}}}{{g}_{algae}} \qquad ( 21 )$$

Based on these assumptions, the rate of CO₂ consumption and O₂ production may be expressed in terms of the growth rate. In particular, the mass production and consumption rates of CO₂ and O₂, respectively, are

\dot{m}_{CO}_{₂₍}g/L)=1.83\dot{m}_{algae} (22)

\dot{m}_{O}_{₂₍}g/L)=1.08\dot{m}_{algae} (23)

In general, the relationships may be expressed as

\dot{m}_{CO}_{₂}=K_{CO}_{₂}\dot{m}_{algae} (24)

\dot{m}_{O}_{₂}=K_{O}_{₂}\dot{m}_{algae} (25)

where, KCO₂ and KO₂ are the amount of gas consumed/produced per mass of microalgae growth and may be in units other than grams gas per grams microalgae. An example of this is set forth below in the controller section, in which the amount of CO₂ is measured in standard liters per minute (SLPM). Where V_{PBR(L)} is the volume of the PBR in liters and assuming that there are 1.808 g_{CO}_{₂} per standard liter (SL), then K_{CO}_{₂} may be expressed as

$$\begin{matrix}{\dot{m}}_{{CO}_{2}} = \frac{1 h}{60 min} \frac{1 SL}{1.808 {g}_{{CO}_{2}}} {V}_{PBR ( L )} 1.83 \frac{{g}_{{CO}_{2}}}{{g}_{algae}} {\dot{m}}_{algae ( g / L / h )} \\ = \frac{1.83 {V}_{PBR ( L )}}{108.48} {\dot{m}}_{algae ( g / L / h )}\end{matrix} \qquad ( 26 )$$

For some photobioreactors according to embodiments of the present invention, the panel dimensions are: 11 inches (hₘ=0.2794 m) tall, 1.5 inches (wₘ=0.0381 m) thick, and 50 feet (lₘ=15.24 m) long. Since there are two panels for each reactor, a single reactor is holding V_{PBR(L)}=324.4658 L of media. This means that the CO² consumption rate is \dot{m}_{CO}_{₂}_{(SLPM)}=5.4736\dot{m}_{algae(g/L/h)}.

The units and description for system parameters for the growth model are given in Table 1.

TABLE 1
List of Growth Model Parameters

| Variable | | |
|---|---|---|
| Name | Units | Description |
| I_{PAR} | mol | Incident Light from Sun (External Input) |
| | photons/m² | |
| u_{D} | g/L/h | Dilution Rate (Commanded Input) |
| ${u}_{{\dot{m}}_{{CO}_{2}}}$ | SLPM CO₂ | CO₂ Mass Flow Rate (Commanded Input) |
| m_{algae} | g/L | Dry Mass (DM) |
| \dot{m}_{algae} | g/L/h | DM Growth Rate |
| K_{PAR} | m²/mol light | Sun Utilization (Constant) |
| R | 1/h | Rate of Respiration in the Dark |
| | | (Constant) |
| \dot{m}_{CO}_{₂} | g_{CO}_{₂}/h | CO₂ Mass Consumption Rate |
| K_{CO}_{₂} | g_{CO}_{₂}/(g_{algae}/L) | CO₂ Biofixation Activity (Mass CO₂ per |
| | | DM) |
| \dot{m}_{O}_{₂} | g_{O}_{₂}/h | O₂ Mass Production Rate |
| K_{O}_{₂} | g_{O}_{₂}/(g_{algae}/L) | O₂ Production Activity (Mass O₂ per DM) |

In general, the growth rate may be a function of available light photons, available nutrients, dissolved CO₂, dissolved O₂, temperature, and media recipe (e.g., media pH). All of these can both be included in the model parameters considered here and be modeled as separate terms, according to embodiments of the present invention.

### 1.1.3 Water Chemistry Subsystem

The water chemistry subsystem models both the dissolved gases and nutrients available to the microalgae in the media, according to embodiments of the present invention. The dissolved gases are a function of both the gases being delivered from an external source and the internal gases being consumed and generated by the microalgae. The external source may provide a constant flow rate of gas, a pattern of turning the constant flow on and off, or a continuously variable flow rate and variable mixture of different gases, according to embodiments of the present invention.

One reason for the use of sparging is to regulate the concentrations of dissolved O₂ and dissolved CO₂ through mass transfer. In general, the gas transfer rates may be modeled locally as a first order dynamic system. Due to the distributed nature of the system, the model may employ many cascaded first order systems, which is common with process models. This phenomenon may be essentially captured by using a first order plus dead time model, which is the method described below. When the media in the PBR is at equilibrium with air, there is about 7 mg/mL of dissolved O₂ in the media, which is maintained through sparging when there is no growth. During high growth periods, dissolved O₂ will build up in the system and is eventually purged at night. This is described by the following dynamic model.

$${\dot{m}}_{DO} ( t ) = \frac{{w}_{sparge}}{{τ}_{DO}} ( {m}_{DO , gas} ( t - {τ}_{d , gas} ) - {m}_{DO} ) + {\dot{m}}_{{O}_{2}} ( t )$$

Here, w_{sparge} is the flow rate of gas into the PBR, τ_{DO} is the lag time for mass transfer of DO between the media and sparging bubbles, m_{DO,gas} is the DO level to which the media will equilibrate, and \dot{m}_{O}_{₂} is the rate of oxygen produced through photosynthesis. When sparging is turned off (i.e., w_{sparge}=0), then DO will build up in the system at the rate that it produced by photosynthesis. Once sparging is turned back on, the DO levels will equilibrate back to m_{DO,gas} with a lag time of

$$\frac{{τ}_{DO}}{{w}_{sparge}} .$$

The input gas stream is an air plus CO₂ gas stream for which the amount of added CO₂ varies. This variation may change the equilibrium valve m_{DO,gas}. There is a delay from when the CO₂ concentration changes and when the new gas mixture arrives at the media, which is captured by the delay τ_{d,gas}. For the model employed by embodiments of the present invention, it may be assumed that m_{DO,gas}=7/mg/mL, independent of the CO₂ concentration.

A similar method may be used to model the dissolved CO₂ according to embodiments of the present invention. This is given by

$${\dot{m}}_{DI C} ( t ) = \frac{{w}_{sparge}}{{τ}_{DIC}} ( {m}_{DIC , gas} ( t - {τ}_{d , gas} ) - {m}_{DIC} ) + {\dot{m}}_{{CO}_{2}} ( t )$$

Here, m_{DIC,gas} is the CO₂ gas concentration required for a specific pH. As CO₂ is removed from the media through photosynthesis (i.e., \dot{m}_{CO}_{₂}), the value of m_{DIC,gas} will be increased to help replace the consumed CO₂. Therefore, this value is always changing during active growth to maintain a constant pH. Due to the distributed nature of the system, there is a delay of τ_{d,gas} between when the commanded CO₂ concentration changes and when the CO₂ reaches the media.

As CO₂ dissolves in the media, it breaks down into different species, namely carbonic acid, bicarbonate, and carbonate. The addition of dissolved CO₂ decreases the pH in the media and the concentration of each of the carbon species in turn depends on the pH. Because it takes a few seconds for carbon to dissolve and only a fraction of the input CO₂ dissolves before leaving the vent, there are some dynamics associated with the pH in the media, according to embodiments of the present invention. According to some embodiments of the present invention, such dynamics can be accounted for by the first order dynamics (transfer function)

$$pH ( t ) = \frac{1}{{τ}_{p H}} ( {K}_{p H} {m}_{DIC} ( t ) - pH ( t ) )$$

Here, τ_{pH} is the lag time associated with the DIC settling into the appropriate species and K_{pH} is the conversion factor from DIC to pH units, according to embodiments of the present invention. Therefore, the control objective is to deliver CO₂ to the location of photosynthetic activity at the rate that it is being consumed, which is the basis of the FF predictive controller, according to embodiments of the present invention. The pH model was linearized about the pH of the media when there was no CO₂ being delivered, according to embodiments of the present invention.

CO₂ is not the only factor affecting pH. Others have found that the pH is also affected by calcium carbonate precipitation in the media and nitrogen assimilation, excess cation influx, excess anion flux, and organic assimilation and excretion by the microalgae. However, the main controllable and measurable variable is CO₂ input which has the most significant effect on pH. This characteristic creates some challenges involving the separate control of CO₂ and pH.

Temperature, pressure, gas flow rate, bubble size, pumping, media recipe and PBR geometry (e.g., panel thickness and height) may also affect the amount of dissolved gases and pH. In one embodiment, these terms are grouped into efficiency parameters, but they may also be modeled as separate components.

### 1.2 FF/FB Control Methodologies

### 1.2.1 FF/FB Control Methodology #1

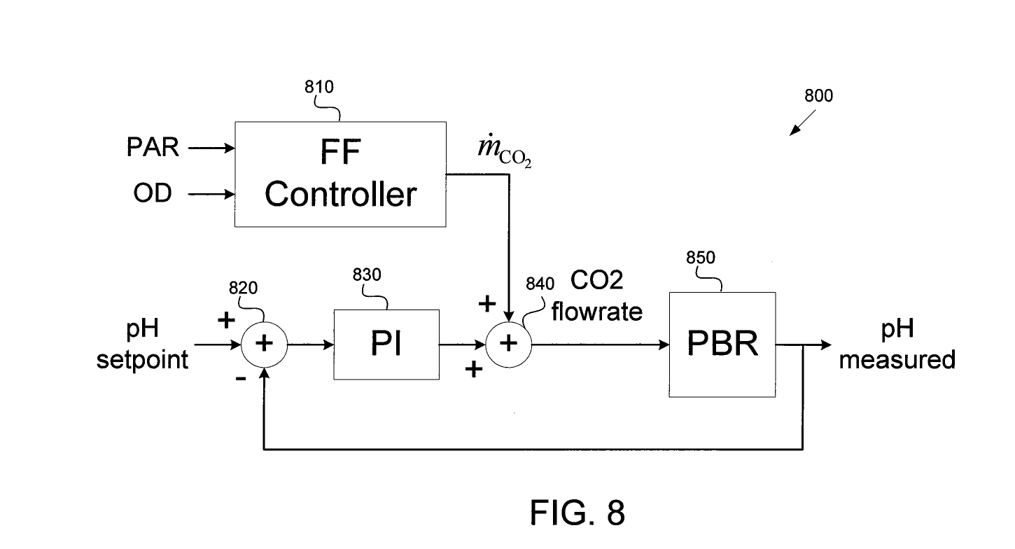

FIG. 8 is a block diagram 800 illustrating the use of a feedforward controller 810 plus a feedback controller to regulate pH via CO₂ addition to photobioreactor 850 in accordance with one or more embodiments of the present invention. In the embodiments illustrated in FIG. 8, feedforward controller 810 reads in the PAR and OD/dry mass from sensors. The FF controller 810 calculates a feedforward commanded CO₂. This FF command is added at summer 840 to the feedback command from the feedback controller 830 and sent to the PBR 850. The pH measured from the PBR 850 is fed back and compared at summer 820 to the pH setpoint 820. The error between the desired and actual pH is sent to the feedback controller 830. According to various embodiments, feedback controller 830 is a lead compensator, lead/lag compensator, a proportional (P) controller, a proportional and integral (PI) controller, a proportional, integral and differential (PID) controller, or other type of controller.

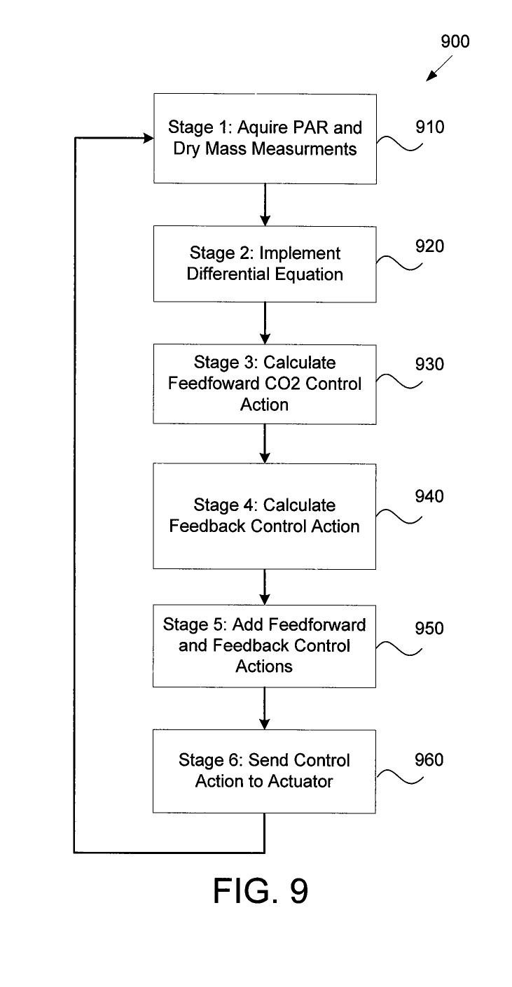

FIG. 9 is flowchart 900 showing an exemplary set of operations for using a feedforward controller plus a feedback controller to regulate pH via CO₂ addition to a photobioreactor in accordance with various embodiments of the present invention. As illustrated in the embodiments shown in FIG. 9, flowchart 900 can be split into 6 stages with different operations. Stage 1 includes reading operation 910 to read in the PAR and OD/dry mass signals. Stage 2 includes implementation operation 920 to implement the growth model differential equations. These equations may either be open loop or observer corrected differential equations. Stage 3 includes calculation operation 930 to calculate the FF CO₂ commanded flow rate. Using the flow rate, stage 4 uses control generation operation 940 to calculate the feedback commanded CO₂ flow rate. Stage 5 uses addition operation 950 to add the two commanded CO₂ flow rates together. Stage 6 uses communication operation 960 to send the total commanded CO₂ flow rate to the actuators. In some embodiments, once communication operation 960 is completed, the method returns to reading operation 910 in stage 1.

In some embodiments, an observer is configured to detect a sensed condition of one or more environmental conditions over time and generate an observer signal indicating the sensed condition. A correction unit is configured to receive the observer signal. In some embodiments, the correction unit updates the algal growth model based on the observer signal.

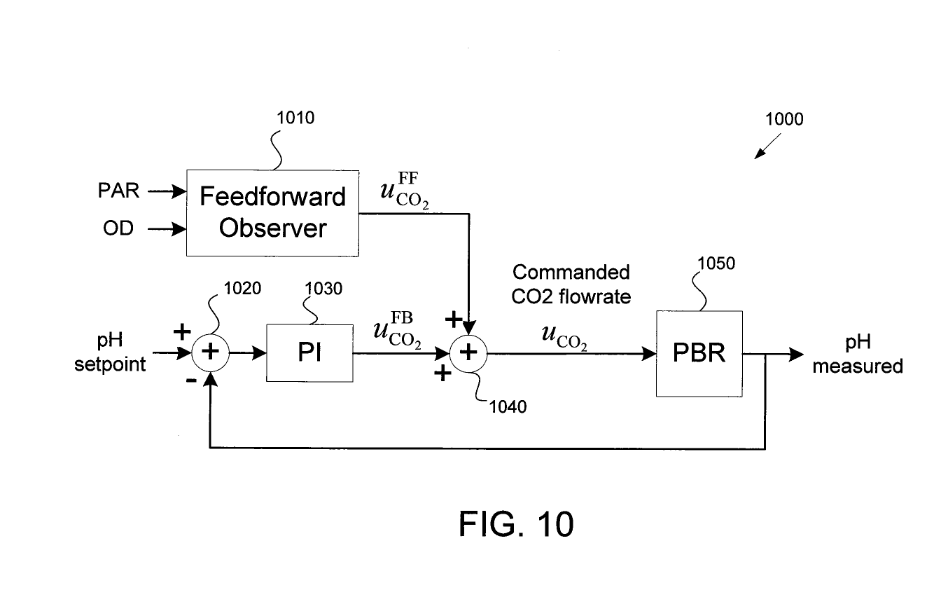

FIG. 10 illustrates a system 1000 for controlling photobioreactor 1050 with feedforward controller 1010 and feedback controller 1030 using an observer corrected model in accordance with one or more embodiments of the present invention. The objective of the controller illustrated in the embodiments shown in FIG. 10 is to maintain a specified pH by delivering CO₂ as the algae consume it through photosynthesis. The feedforward controller 1010 takes external measurements, namely PAR and measured OD/dry mass, and calculates the feedforward CO2 control signal u_{CO}_{₂}^{FF}. A measurement of the pH can be taken, or estimated, from PBR 1050 (e.g., using one or more sensors) and compared to the desired pH in summer 1020. Based on the error signal generated from summer 1020, feedback controller 1030 can generate a feedback correction, namely u_{CO}_{₂}^{FB}, which can be added using summer 1040 to the feedforward correction, namely u_{CO}_{₂}^{FF}, to produce the commanded CO₂ flowrate, namely u_{CO}_{₂}, that is sent to the CO₂ actuator on the PBR 1050.

### 1.2.2 Observer Based Control

Observers are used to estimate the internal states of the photobioreactor system, according to embodiments of the present invention. This state estimate may be used for state feedback controller implementation; however, the state estimate may also be used to do predictive feedforward control, according to embodiments of the present invention. The state-space representation of a model is:

\dot{x}=Ax+Bu

y=Cx+Du (29)

An estimate of the state variable x is created by explicitly creating the model in equation (29). This estimated state variable is labeled \dot{x}, and the estimated output that it produces is \dot{y}. The state space equations for the state estimator are:

{circumflex over (\dot{x}=A\hat{x}+Bu

ŷ=C\hat{x}+Du (30)

The actual input to the system, u, is used to produce these estimates, according to embodiments of the present invention.

In order to track the state x, the error signal between the estimated output ŷ and the measured (actual) output y is used to correct the state estimate \hat{x}. With this feedback, the closed loop model of \hat{x} is

$$\begin{matrix}{x}^{\dot{\hat{}}} = A \hat{x} + Bu + L ( \hat{y} - y ) \\ = A \hat{x} + Bu + L [ ( C \hat{x} + Du ) - ( Cx + Du ) ] \\ = A \hat{x} + Bu + LC ( \hat{x} - x )\end{matrix} \qquad ( 31 )$$

Next, the error signal between the estimated state and actual state is defined as

e=\hat{x}−x

ė={circumflex over (\dot{x}−\dot{x} (32)

With the help of equations (29) and (31), equation (32) becomes

$$\begin{matrix}\dot{e} = {x}^{\dot{\hat{}}} - \dot{x} \\ = [ A \hat{x} + Bu + LC ( \hat{x} - x ) ] - [ Ax + Bu ] \\ = ( A + LC ) e\end{matrix} \qquad ( 33 )$$

By placing the eigenvalues of A+LC suitably in the open left half plane, it may be guaranteed that this error will be globally asymptotically stable (e.g., \hat{x} will track x). If this observer were to be used for a state feedback controller, then \hat{x} should track x about 5 to 10 times faster than the plant dynamics. Having the same observer bandwidth would be beneficial for the feedforward controller, according to embodiments of the present invention.

### 1.2.3 Observer Based Feedforward Control

The open loop growth model from Section 1.1.2 can be used to model the growth rate and hence the CO₂ consumption rate, according to embodiments of the present invention. If the open loop model is exact, then the FF model perfectly predicts the correct amount of consumed CO₂ according to embodiments of the present invention. However, if there is even the slightest mismatch between the model and the physical system, then the modeled output may eventually diverge away from actual output. This can happen where simple models (e.g., first order models) are used to model very complex systems.

To deal with this, an observer may be added to the growth model that will correct for model differences and help track the growth rate, according to embodiments of the present invention. The derivation of an observer for a Linear Time Invariant system was derived in the previous section along with a proof of global convergence, according to embodiments of the present invention. For the nonlinear growth model presented in Section 1.1.2, the same technique may be successfully applied; however, the statements of global convergence may no longer hold. However, the system can be made stable by an appropriate choice of the observer gain L.

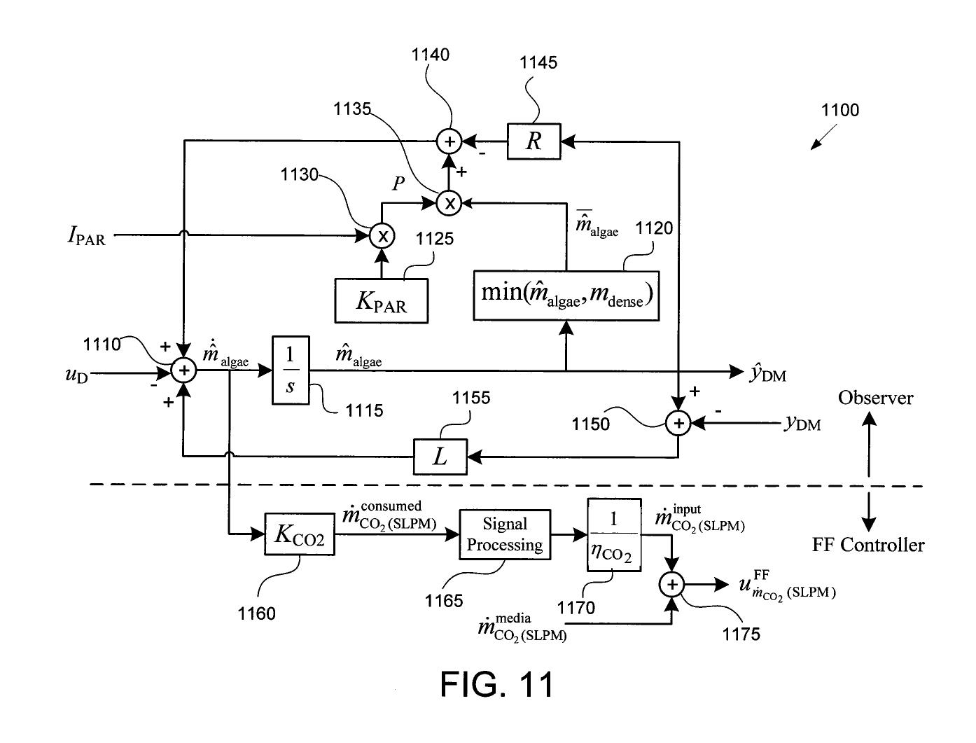

FIG. 11 is a block diagram 1100 showing an example of an observer corrected growth model that may be used as a feedforward pH controller in accordance with some embodiments of the present invention. In addition to the observer, a FF CO₂ controller may also be used, as shown in FIG. 11, according to embodiments of the present invention. As with the observer derived in the previous section, the estimated growth rate and dry mass variables are denoted by “hats”.

The differential equation describing the observed {circumflex over (\dot{m}_{algae} is

{circumflex over (\dot{m}_{algae}=K_{PAR}I_{PAR}{circumflex over ( m̅_{algae}−R\hat{m}_{algae}−u_{D}+L(ŷ_{DM}−y_{DM})

ŷ_{DM}=\hat{m}_{algae} (34)

This is calculated by multiplying K_{PAR} 1125 by the input I_{PAR} from the light model subsystem in 1130. The output of 1115 is saturated in 1120 to form {circumflex over ( m̅_{algae}. The productivity parameter P=K_{PAR}I_{PAR}{circumflex over ( m̅_{algae} algae is formed by multiplying (e.g., using multiplier 1135) the outputs from 1120 and 1130. The term R\hat{m}_{algae} is formed in block 1145, which is then subtracted from P using summer 1140. The output of summer 1140 is fed back to summer 1110. In the observer correction path, the measured dry mass y_{DM} is subtracted 1150 from the estimated dry mass ŷ_{DM}. This error is multiplied 1155 by the observer gain L and is fed back to 1110. The outputs of 1155 and 1140 are added and a harvest/dilution rate is subtracted 1110 to form the estimate of the dry mass growth rate {circumflex over (\dot{m}_{algae}. This is integrated 1115 to form the estimate of the dry mass \hat{m}_{algae} (or ŷ_{DM}).

The observer part of the controller in FIG. 11 is contained above the dashed line and the FF controller is contained below the dashed line. For the FF control parameters, K_{CO}_{₂} 1160 is chosen such that it models the amount CO₂ consumed in the appropriate units. For the purposes of control, this will generally be in units of SLPM. The growth rate signal may be very noisy. To account for this, signal processing module 1165 (e.g., low pass filtering or moving averaging) may be used, according to embodiments of the present invention. Then, an efficiency parameter, namely η_{CO2}_{₂} 1170 may be fit from data. This efficiency is the ratio of CO₂ consumed to input CO₂. This efficiency divides the consumed CO₂ to arrive at the amount of input CO₂ required for the current growth rate, namely m_{CO2}_{₂}^{input}. Also, there is an amount of input CO₂ to keep the media pH balanced, which is represented by the parameter u_{CO2}_{₂}^{media}. The addition of u_{CO2}_{₂}^{media} and m_{CO2}_{₂}^{input} make up the FF CO₂ control signal u_{CO2}_{₂}^{FF} 1175, according to embodiments of the present invention.

The term y_{DM} represents the measured dry mass from the turbidity sensor that is used to form the observer correction. While this is one example of a correction that may be implemented, based on the disclosure provided herein one of ordinary skill in the art will appreciate that other measurements may be used to correct the model, according to embodiments of the present invention. For example, a pH reading may be used to correct the model. Other less expensive measurements may be used to control the model, according to embodiments of the present invention.

A continuously updating observer (in which the observer is always updating) is described, above, according to embodiments of the present invention. Instead of a continuously updating observer, an operator could take a measurement periodically (e.g. once a day) that either gives a correction to the model or resets the integrator to the correct value, according to embodiments of the present invention. In either case, the model can run open loop until another correction is applied, according to embodiments of the present invention.

Assuming that \hat{m}_{algae}<m_{dense} so that \hat{m}_{algae}={circumflex over ( m̅_{algae}, then a nonlinear time varying (“NLTV”) state space representation of the system with two inputs, one output, and one state may be given by

$$\begin{matrix}{{m}^{\dot{\hat{}}}}_{algae} = {A}_{g} ( {I}_{PAR} ) {m}_{algae} + {B}_{g} {u}_{g} \\ {\hat{y}}_{DM} = {C}_{g} {\hat{m}}_{algae} + {D}_{g} {u}_{g} \\ where & ( 35 ) \\ {A}_{g} ( {I}_{PAR} ) = {K}_{PAR} {I}_{PAR} - R \\ {B}_{g} = [ 0 - 1 ] \\ {C}_{g} = 1 \\ {D}_{g} = [ 0 - 1 ] \\ {u}_{g} = [ \begin{matrix}{I}_{PAR} \\ {u}_{D}\end{matrix} ] & ( 36 )\end{matrix}$$

Here, the subscript “g” is used to denote the growth model. A_{g} is time varying and nonlinear, so the observer is not mathematically guaranteed to globally converge, according to embodiments of the present invention. Nonetheless, the same techniques from the linear time-invariant (“LTI”) observer may be applied to the equation 36 to design the gain L. A_{g} and L are scalars and the expression A_{g}+LC_{g}=A_{g}+L may be designed to satisfy A_{g}+L<0 for a class of expected A_{g}.

The following choice may be made, according to embodiments of the present invention:

L<−5*max{A_{g}}. (37)

The maximum A_{g} will occur when the sun is at its peak for the day (e.g., when I_{PAR} is at its maximum value). According to some embodiments of the present invention, the range that A_{g} covers will be too large and there will have to be different L parameters for different ranges of light intensities. Equation 37 could also be used for the cases when \hat{m}_{algae≧m}_{_{dense}}; however, this may result in an overly aggressive observer, according to embodiments of the present invention.

### 1.2.4 FF/FB Control—Additional Implementation

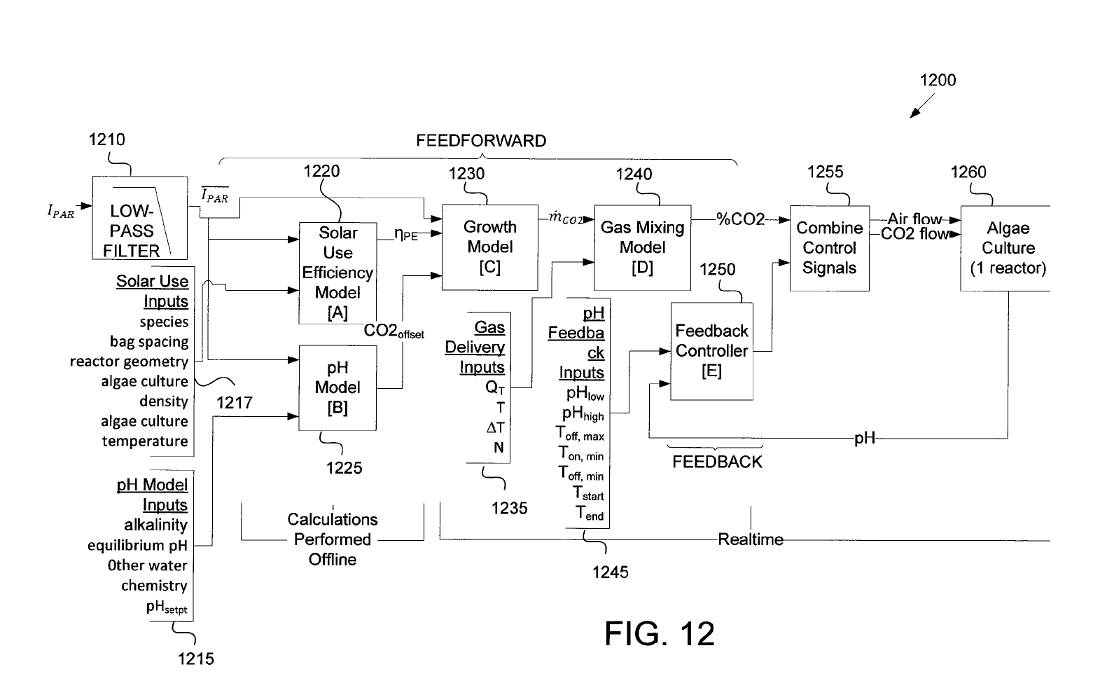

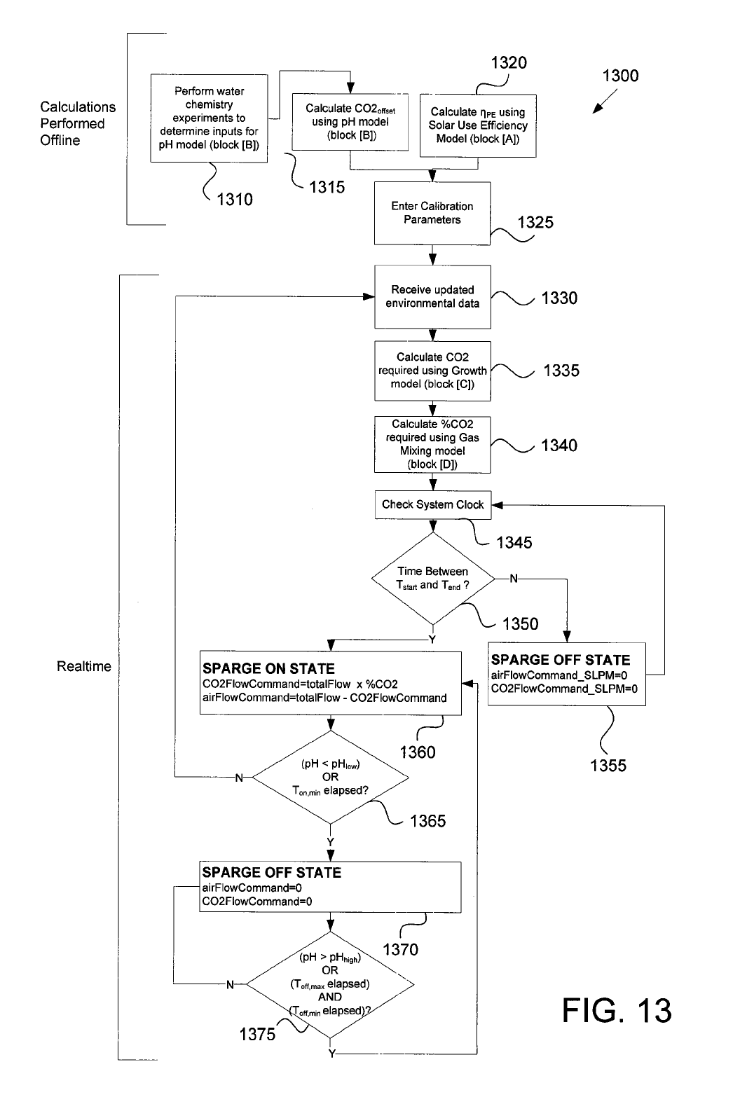

FIG. 12 is a block diagram 1200 illustrating an example of an implementation of a controller for controlling an algae culture using feedforward control and feedback in accordance with some embodiments of the present invention. The embodiments illustrated in FIG. 12 include a set of sub-models (1220, 1225, 1230, 1240) that use environmental and operation parameters 1215 to provide a feedforward prediction of CO2 required, according to embodiments of the present invention. The output of gas mixing model 1240 is directed to combining controller 1255 where signal is combined with the feedback signal from the feedback controller 1250 to deliver the appropriate amount of CO2 to the algae culture 1260. The feedback controller 1250, using pH feedback inputs 1245, and combining controller 1255 works according to the logic described in FIG. 13.

In the embodiments illustrated in FIG. 12, the primary environmental input to the system is solar PAR data, which is filtered by a low-pass filter 1210 before being provided to the sub models 1220 and 1225. The solar use efficiency model 1220 can work as follows. η_{PE} is the primary output of the Solar Use Efficiency Model, and represents photosynthetic efficiency, expressed as grams of biomass per mol of photons incident on a horizontal surface. At the theoretical limit of perfectly efficient photosynthesis, η_{PE} can be calculated from the quantum requirement (photons required to fix one carbon in the basic photosynthesis equation) and energy contents:

$${CO}_{2} + {H}_{2} O + 8 photons -> {CH}_{2} O + {O}_{2}$$

$$\frac{1 mol {CH}_{2} O}{8 mol photons} \times \frac{{E}_{{CH}_{2} O}}{1} \times \frac{1}{{E}_{biomass}} = \frac{g biomass}{mol photons}$$

$$\frac{1 mol {CH}_{2} O}{8 mol photons} \times \frac{482.5 kJ}{mol {CH}_{2} O} \times \frac{g biomass}{26.9 kJ} = 2.2 \frac{g biomass}{mol photons}$$

As it is used here, the actual value (below theoretical) accounts for many efficiencies, including, but not limited to, photon transmission to reach the algae culture, photon capture efficiency by the algae, energy use efficiency of the algae. According to various embodiments, primary inputs 1217 to the model may include one or more of algae culture density, algae culture temperature, algae species, and/or reactor parameters such as geometry and bag spacing. In one implementation of some embodiments of the present invention, this model is empirically based on results from growth data.

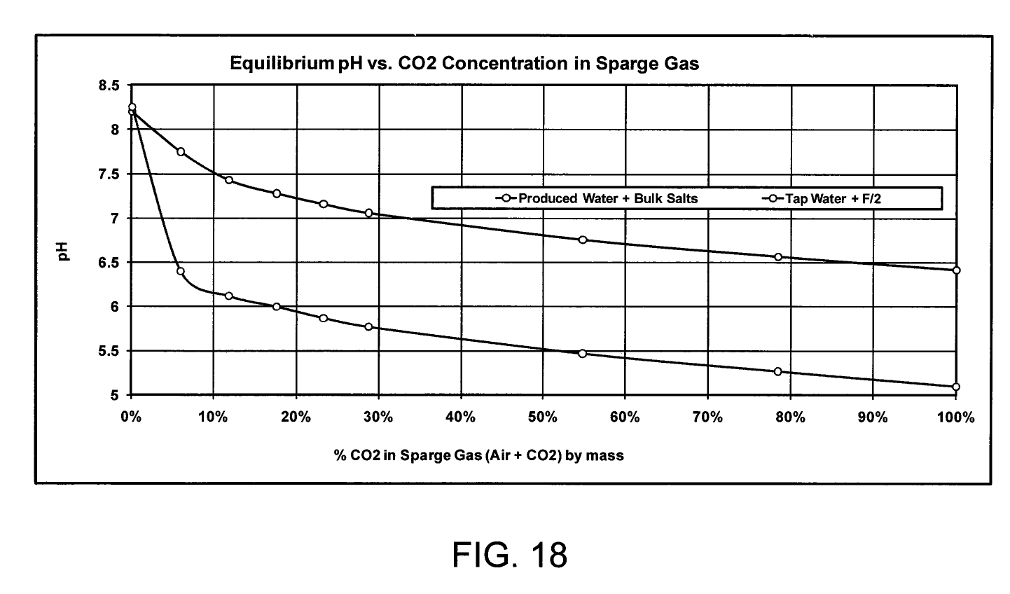

The pH model 1225 works as follows in some embodiments of the present invention. CO2_{offset} is the primary output of the pH model, and represents the amount of CO2 that must be added to the media to achieve the desired pH without algae growth. The equilibrium pH of the water for algae growth will be a function of the inputs 1215 of water and media parameters that may include alkalinity, equilibrium pH, the pH set point, and/or other water chemistry measurements. For example, water with a high concentration of bicarbonate will tend to be buffered, such that more CO2 is required to lower the pH to the same amount as water with a lower concentration of bicarbonate. FIG. 18, described in more detail below, illustrates this point for two media sources. The top curve (“produced water”) has a high bicarbonate concentration relative to the bottom curve (“tap water”).

In one implementation according to embodiments of the present invention, this model can be experimentally based on results from data. The growth model 1230 produces the output of \dot{m}_{CO2} with the following equation

$${\dot{m}}_{CO 2} = \frac{\overline{{I}_{PAR}}}{{10}^{6}} \cdot A \cdot \frac{{η}_{PE}}{1 - R} \cdot \frac{{C}_{algae}}{{C}_{CO 2}} + CO {2}_{offset}$$

$$Where \text{:}$$

$${\dot{m}}_{CO 2} = instantaneous use of CO 2 for photosynthesis ( g \text{/} s )$$

$$\overline{{I}_{PAR}} = rate of photons incident on the system ( μmol \text{/} {m}^{2} \text{/} s ) ,$$

which when multiplied by A, horizontal area (m²), and divided by 10⁶, is converted to mol/s. I̅_̅{̅P̅A̅R̅}̅ is processed from I_{PAR} via a low-pass filter. Area is defined as the horizontal area of photon capture. η_{PE}=photosynthetic efficiency, the ratio of net algae biomass accumulated to incident photons (g/mol), explained above. R=ratio of nighttime biomass loss to daytime biomass accumulation. This term is included because photosynthetic efficiency has been primarily calculated based on net biomass accumulation, but CO2 is delivered during the daytime (for a daytime-only sparge scheme). Thus, the daytime-equivalent value is found by

$$\frac{{η}_{pE}}{1 - R} .$$

For example, if daytime growth is 1 g, and subsequent nighttime loss is 0.3, then the net growth rate would be 0.7, and R would be 0.3/1=0.3. The value of R is 0.3 on average from experimentally grown algae batches over a certain period of time. C_{algae}=portion of algae that is carbon, by mass, which may be taken to be 0.50. C_{CO2}=portion of CO2 that is carbon, by mass, =0.273, based on atomic weights of carbon and oxygen (12 and 16, respectively):

$$\frac{{M}_{C}}{{M}_{CO 2}} = \frac{12}{( 12 + 2 \times 16 )} = 0.273$$

CO2_{offset}=the amount of CO2 that must be added to the media to achieve the desired pH without algae growth, explained above.

The growth sub-model can output either \dot{m}_{CO2}, as described in the equation as stated above, or \dot{m}_{algae}, the instantaneous rate of algae biomass increase (g/s):

$${\dot{m}}_{algae} = \frac{\overline{{I}_{pAR}}}{{10}^{6}} \cdot A \cdot \frac{{η}_{pE}}{1 - R}$$

Therefore \dot{m}_{CO2} can also be expressed as

$${\dot{m}}_{CO 2} = {\dot{m}}_{algae} \cdot \frac{{C}_{algae}}{{C}_{CO 2}} + CO {2}_{offset}$$

The gas mixing model 1240 produces the output of % CO₂ with the following equation, where inputs for the equation are provided by gas delivery inputs 1235:

$$\% CO 2 = \frac{{\dot{m}}_{CO 2}}{{ρ}_{CO 2}} \cdot \frac{T}{Δ t} \cdot \frac{1}{{Q}_{T}}$$

For a gas delivery scheme that is intermittent rather than continuous, the CO2 will be delivered in shorter time windows as shown in FIG. 18, and therefore, higher concentrations. Where:

% CO2=portion of the total flow, by mass, that is CO2 (represented as actual value, not percent)

ρ_{CO2}=density of CO2 at STP (=1.97 g/L)

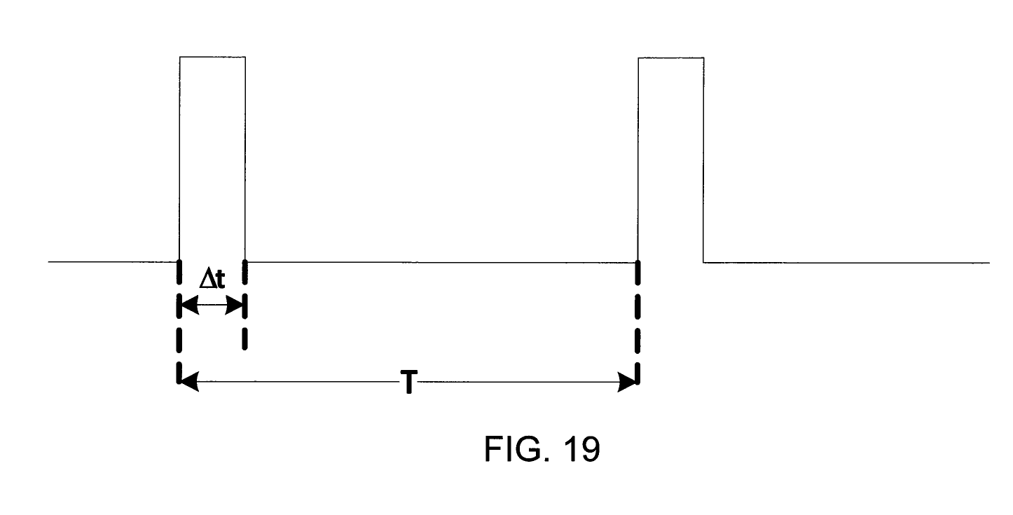

T=Total time between start of 2 consecutive sparge events (seconds) as illustrated in FIG. 19.

Δt=length of time of one sparge event (seconds) as illustrated in FIG. 19.

Note that the ratio Δt/T is the duty cycle (D), and its value will have a lower limit driven by mixing requirements (e.g. culture circulation, dissolved O₂ removal). The value of T can be adjusted as a parameter and is largely driven by the desired pH range (and also by mixing requirements).

Q_{T}=total desired gas flow (SLPM).

An additional parameter that will be implemented in this model is N, the number of gas exchanges of the space above the algae culture in the bag intended for each sparge event.

FIG. 13 is a flowchart 1300 showing an example of a set of operation for the implementation of a controller for controlling an algae culture using feedforward control and feedback in accordance with one or more embodiments of the present invention. This implementation of a controller using feedforward control combined with feedback, which is in accordance with some embodiments, can be implemented in two phases. In some cases, the two phases will be an offline portion and an online portion.

The portion performed offline consists of a pH model 1310 and 1315, which corresponds to 1225 and the solar use efficiency model 1320, which corresponds to 1220. The outputs of these sub models provide calibration parameters, which are entered in the entering calibration parameters step 1325. The real-time portion receives these calibration parameters once at the inception of the program and receives updated environmental data 1330 continuously via the loop returning to this step 1330 from 1365. Each time updated environmental data is received (1330), in the next step 1335 these inputs are used by the growth model to calculate CO2 required, which corresponds to 1230. In the next step 1340, gas mixing model uses CO2 required to calculate % CO2, which corresponds to 1240.

In steps 1345 and 1350, the time of day is checked against start and end time limits to determine whether the sparging will be commanded to be off in state 1355 (usually during nighttime) or controlled on/off (usually during daytime). If the system clock time is between the time limits, sparging is turned on in state 1360, where the CO2 and air flows are commanded according to % CO2 supplied by the feedforward sub-models (1330, 1335, and 1340). The system then enters a feedback loop, by checking the sensed pH value against a lower limit and a minimum time setting in 1365. If the conditions are not met, the system loops back to receive updated environmental parameters in step 1330. If the conditions are met, the system goes to a sparge off state 1370 where sparging is commanded off and checks the sensed pH value against an upper limit and two additional time settings in step 1375. If the conditions in step 1375 are not met, the system loops back to state 1370 until the conditions are met. If the conditions in step 1375 are met, the system returns to the sparge on state 1360.

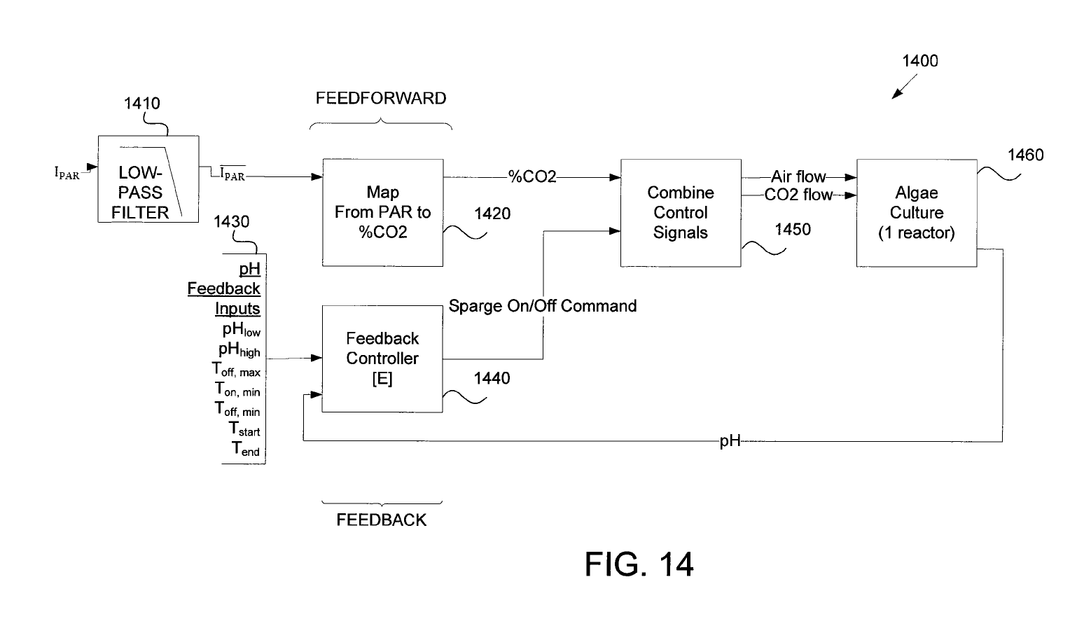

FIG. 14 illustrates a block diagram 1400 showing an example implementation of a controller using feedforward control combined with feedback in accordance with one or more embodiments of the present invention. This is a generalized implementation of a controller using feedforward control combined with feedback, very similar to the implementation described in FIG. 12, except the details of the step of mapping from PAR to % CO2 are not detailed by sub models, but instead happens via a configurable static equation.

The description for FIG. 14 is comparable to that of FIG. 12 for the components that are in common: the low-pass filter 1410 behaves similarly to 1210, the feedback controller 1440 receiving pH feedback inputs 1430 behaves similarly to 1250 receiving inputs 1245, the combining controller 1450 behaves similarly to 1255, the algae culture 1460 behaves similarly to 1260. The feedforward map from PAR to % CO₂ 1420 consists of a fixed equation, determined experimentally, according to embodiments of the present invention. For one particular successful implementation of such a map, the equation used was % CO2=0.00833× I̅_̅{̅P̅A̅R̅}̅10, where I̅_̅{̅P̅A̅R̅}̅ is in units of umol/m²/s and represents a 15 minute average of I_{PAR}.

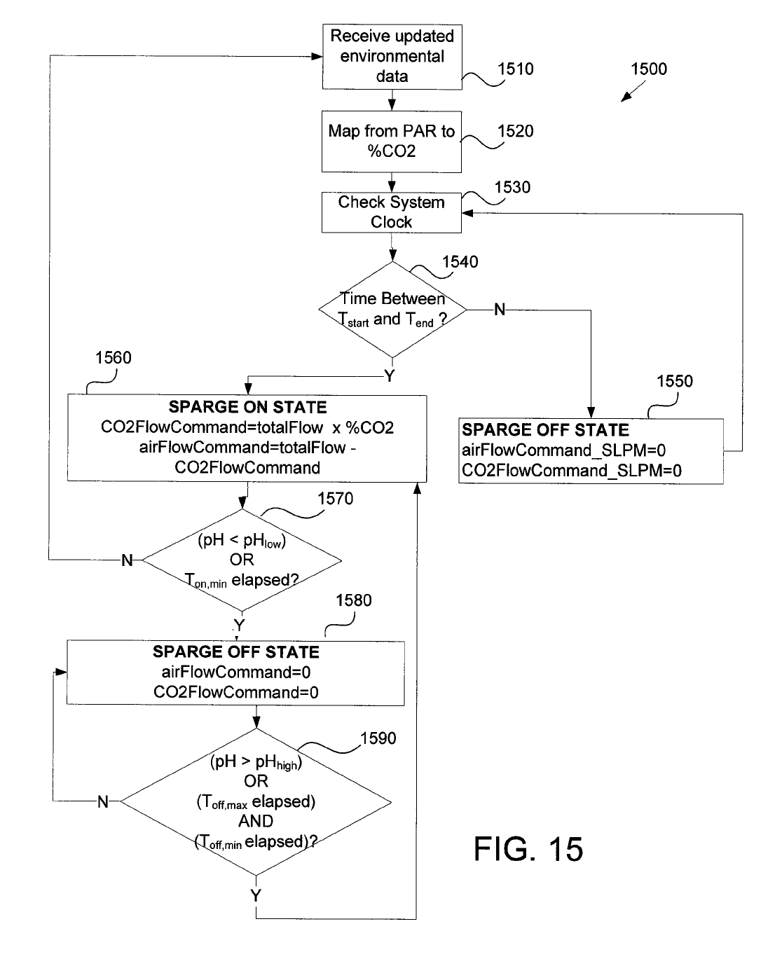

FIG. 15 is a flow chart 1500 illustrating an example of a set of operations for the implementation of a controller using feedforward control combined with feedback in accordance with various embodiments of the present invention. The operations set forth in flowchart 1500 provide a generalized implementation of a controller using feedforward control combined with feedback, very similar to the implementation described in FIG. 13, except the details of the step of mapping from PAR to % CO₂ are not detailed by sub models.

The description for FIG. 15 is similar to that of FIG. 13 for the components that are present: the receive updated environmental data step 1510 behaves similarly to 1330, the map from PAR to % CO₂ step 1520 may use a model, as in the implementation of 1700, or may use a static map, the check system clock step 1530 behaves similarly to 1345, the check time step 1540 behaves similarly to 1735, the sparge off state 1550 behaves similarly to 1355, the sparge on state 1560 behaves similarly to 1360, the feedback checks 1570 behave similarly to 1365, the sparge off state 1580 behaves similarly to 1370, and the other set of pH checks 1590 behaves similarly to 1375.

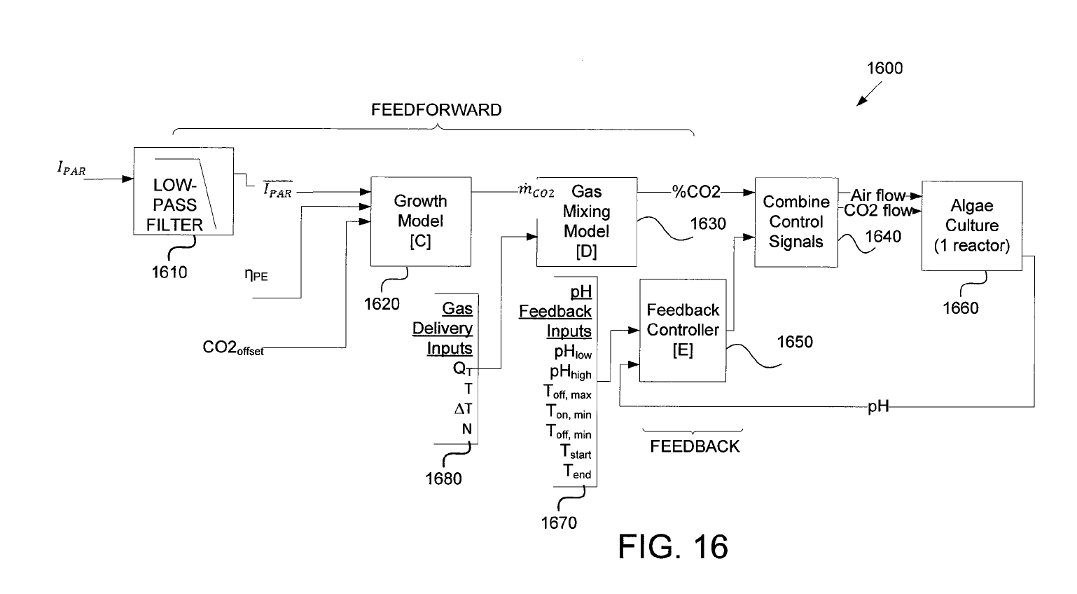

FIG. 16 illustrates a block diagram 1600 with an example of a gas control system with static input parameters to a growth model in a feedforward component in accordance with various embodiments of the present invention. The embodiments shown in FIG. 16 show one implementation of a controller using feedforward control combined with feedback, very similar to the implementation described in system 1200, except two inputs are provided as static inputs which are fixed rather than derived by a model: η_{PE} and CO2_{offset}. The growth model 1620 uses the static inputs and signal from the low pass filter 1610 to estimate amount of CO₂ required. The description for 1600 is similar to that of 1200 for the components that are present. The low pass filter 1610 behaves similarly to 1210, the gas mixing model 1630 using gas delivery inputs 1680 behaves similarly to 1240 using gas delivery inputs 1235, the feedback controller 1650 using pH feedback inputs 1670 behaves similarly to 1250 using pH feedback inputs 1245, the combining controller 1640 behaves similarly to 1455, and the algae culture 1660 behaves similarly to 1260, according to embodiments of the present invention.

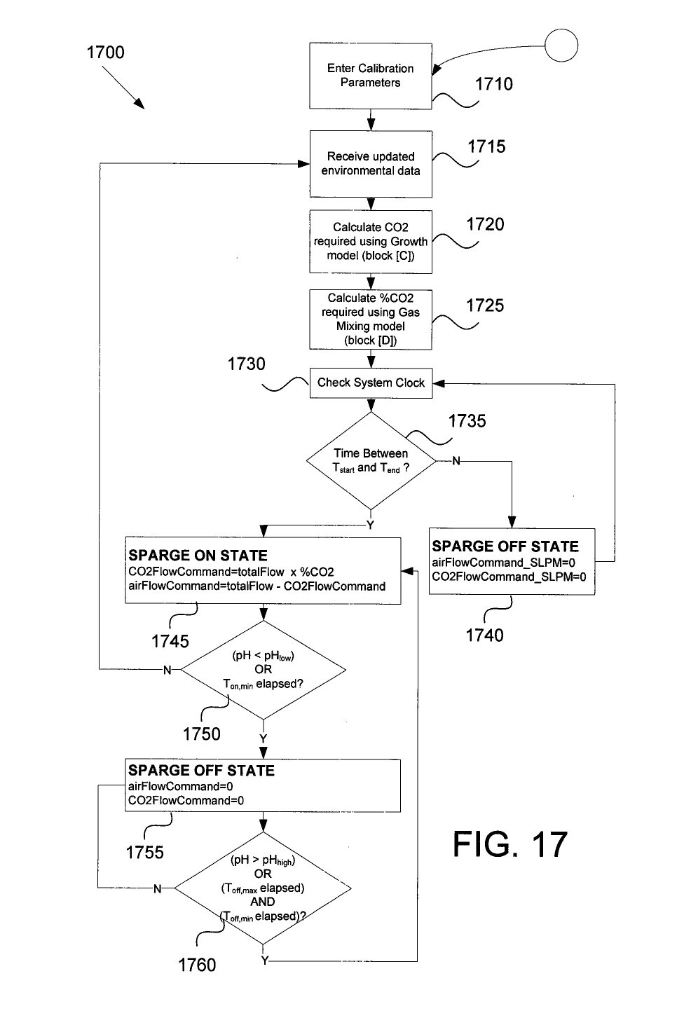

FIG. 17 is a flowchart 1700 illustrating an example of a set of operations for a gas control system with static input parameters to a growth model in a feedforward component in accordance with some embodiments of the present invention. Flowchart 1700 illustrates a specific implementation of a controller using feedforward control combined with feedback, very similar to the implementation described in 1300, except two inputs are provided as static inputs which are fixed rather than derived by a model: η_{PE} and CO2_{offset}.

The description for 1700 is similar to that of 1300 for the components that are present. The enter calibration parameters step 1710 behaves similarly to 1325, the receive updated environmental data step 1715 behaves similarly to 1330, the growth model step 1720 behaves similarly to 1335, the gas mixing model step 1725 behaves similarly to 1340, the check system clock step 1730 behaves similarly to 1345, the check time step 1735 behaves similarly to 1350, the sparge off state 1740 behaves similarly to 1355, the sparge on state 1745 behaves similarly to 1360, the feedback checks 1750 behave similarly to 1365, the sparge off state 1755 behaves similarly to 1370, the other set of pH checks 1760 behaves similarly to 1375, according to embodiments of the present invention.

FIG. 18 is a graph illustrating the equilibrium pH versus carbon dioxide concentration in sparge gas in accordance with various embodiments of the present invention. For a given gas flow rate and media mixture, the % CO₂ in the gas stream will determine the steady state pH value. FIG. 18 shows the steady state value of pH for two different media mixtures. These results are on the media only (i.e., with no algae in the media). FIG. 18 also illustrates that the steady state pH decreases as the % CO₂ increases. FIG. 18 also shows that the steady state pH is dependent on the media used.

FIG. 19 illustrates an example of an intermittent gas delivery scheme in accordance with some embodiments of the present invention. The period between when an ON command may be implemented is given by T. The amount of time that the sparging is on is denoted Δt and the duty cycle fraction is

$$\frac{Δ t}{T} .$$

### 1.2.5 Feedback Control Strategies

According to some embodiments of the present invention, the primary source of feedback control is “bang-bang” and proportional plus integral (“PI”) control. Due to the digital nature of the control hardware and software, a discrete-time equivalent PI controller with an anti-windup scheme is used, according to embodiments of the present invention. A basic continuous time transfer function for a PI controller is set forth below.

$$K ( s ) = {K}_{p} + \frac{{K}_{i}}{s} = \frac{{K}_{p} s + {K}_{i}}{s} \qquad ( 38 )$$

Let the input error signal to the PI controller be labeled e(t) (with associated transfer function E(s)) and let the PI controller output be labeled u(t) (with associated transfer function U(s)). Then,

$$\frac{U ( s )}{E ( s )} = K ( s ) = \frac{{K}_{p} s + {K}_{i}}{s} . \qquad ( 39 )$$

This leads to the equation

sU(s)=KₚsE(s)+KᵢE(s). (40)

Converting this back to a time domain differential equation, this yields

\dot{u}(t)=Kₚė(t)+Kᵢe(t), (41)

which may be expressed as

$$u ( t ) = {K}_{p} e ( t ) + {K}_{i} {\int }_{0}^{t} e ( τ ) d τ . \qquad ( 42 )$$

This is one way of expressing a PI controller because the current output is a scaled (by Kₚ) version of the current error (e.g., the proportional term) plus a scaled (by Kᵢ) history of all of the previous errors (e.g., the integral term). However, for discrete-time implementation, the derivation starts with equation 41. For a simple first order system (like a PI controller), a first order backwards Euler approximation works well. If T is the time between samples, then equation 41 may be discretized as follows:

$$\begin{matrix}\frac{u [ k ] - u [ k - 1 ]}{T} = {K}_{p} \frac{e [ k ] - e [ k - 1 ]}{T} + {K}_{i} e [ k ] & ( 43 ) \\ u [ k ] = u [ k - 1 ] + {K}_{p} ( e [ k ] - e [ k - 1 ] ) + {K}_{i} T e [ k ] . & ( 44 )\end{matrix}$$

Equation 44 may be implemented numerically on computer with two memory elements, according to embodiments of the present invention. The current output, namely u[k], is the previous output, namely u[k−1] plus a correction term, namely Kₚ(e[k]−e[k−1])+KᵢTe[k], which is based on the current and previous error terms, namely e[k] and e[k−1], respectively, according to embodiments of the present invention.

For anti-windup, a saturation may be included on the right hand side of equation 44, which will limit the control action from growing beyond the actuation limits of the actuator, according to embodiments of the present invention. This is illustrated in the following two equations

$$\begin{matrix}u [ k ] = sat [ u [ k - 1 ] + {K}_{p} ( e [ k ] - e [ k - 1 ] ) + {K}_{i} Te [ k ] ] , \\ where & ( 45 ) \\ sat [ \cdot ] = \{ \begin{matrix}{u}_{ma x} & u [ k ] > {u}_{ma x} \\ u [ k ] & {u}_{ma x} > u [ k ] > {u}_{m i n} \\ {u}_{m i n} & u [ k ] < {u}_{m i n}\end{matrix} & ( 46 )\end{matrix}$$

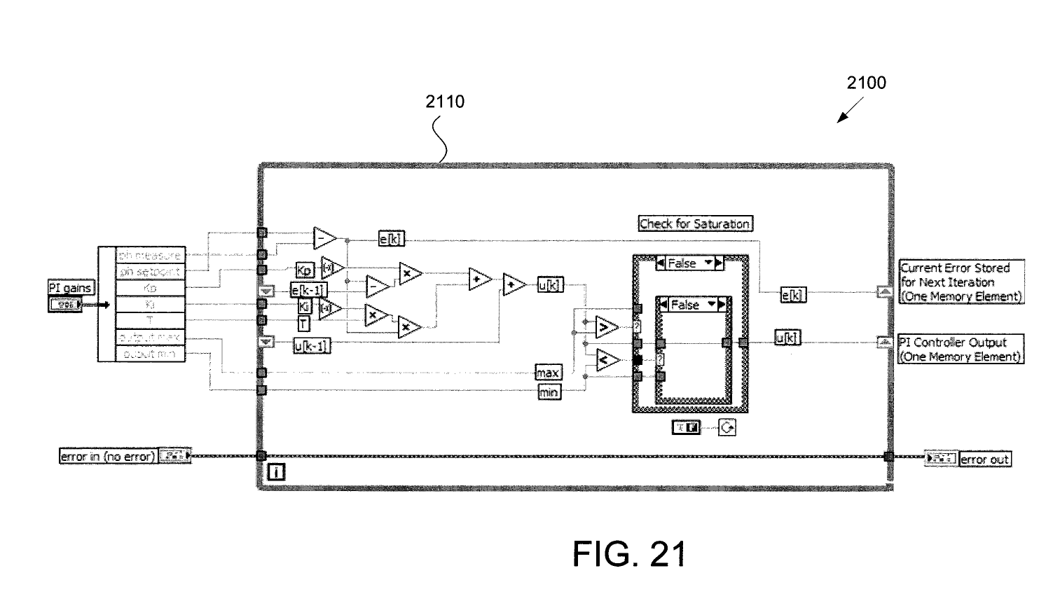

A block diagram of a PI controller used for pH control is shown in FIG. 21, according to embodiments of the present invention. FIG. 21 depicts two memory elements that hold the previously calculated error and control output. In the control (e.g. Labview) software, these memory elements may be shift registers, according to embodiments of the present invention.

### 1.3 Example Applications

Two goals according to embodiments of the present invention are to maximize biomass production and maximize lipid production. In the case of biomass production, the end product could be pharmaceutical products, health food, feed additive, cosmetics, and/or research medicine, for example. In the case of lipid production, the product could be neutral lipids that may be converted into biofuels, for example.

Because simple relationships are often not enough to maximize performance, using dynamic models for photobioreactors provides a significant benefit. For example, it is generally accepted that more sun requires more carbon dioxide (because carbon dioxide is being consumed in the presence of sun), so in such cases the rate of addition of carbon dioxide may be made a direct function of current sun intensity. However, at very low light intensities in the beginning and end part of the day, the amount of algae respirating compared to the amount doing photosynthesis is significant and more carbon dioxide addition may not be necessary at those times. Also, if it has been cloudy for a period of time and then the sun comes out, the algae growth rate will increase dramatically and the amount of additional carbon dioxide required will rise as the algae regain growth momentum. In contrast, if it has been sunny or bright for a period of time, then the amount of required carbon dioxide will not increase as fast because the algae may be experiencing photoinhibition and their growth rate may be limited in other ways. Such effects are best captured through dynamic models and not through static function maps, according to embodiments of the present invention

Another application of the algae growth and/or photobioreactor models is to reduce cost by replacing expensive sensors, according to embodiments of the present invention. Knowledge of bioreactor conditions is helpful for control, but such measurements do not always need to come from sensors, according to embodiments of the present invention. For example, the measurements of cell density, temperature, and pH are helpful for control, but they can be inferred from a model that only monitors sun intensity, according to embodiments of the present invention. In turn, this sun intensity reading may be derived from radiation reading from the Internet that is passed through the “Light Subsystem” to determine the amount of light that the algae experience, according to embodiments of the present invention. According to an alternative embodiment, cell density may be inferred from dissolved O₂ or pH within the media inside the photobioreactor.

### 1.4 Scheduling

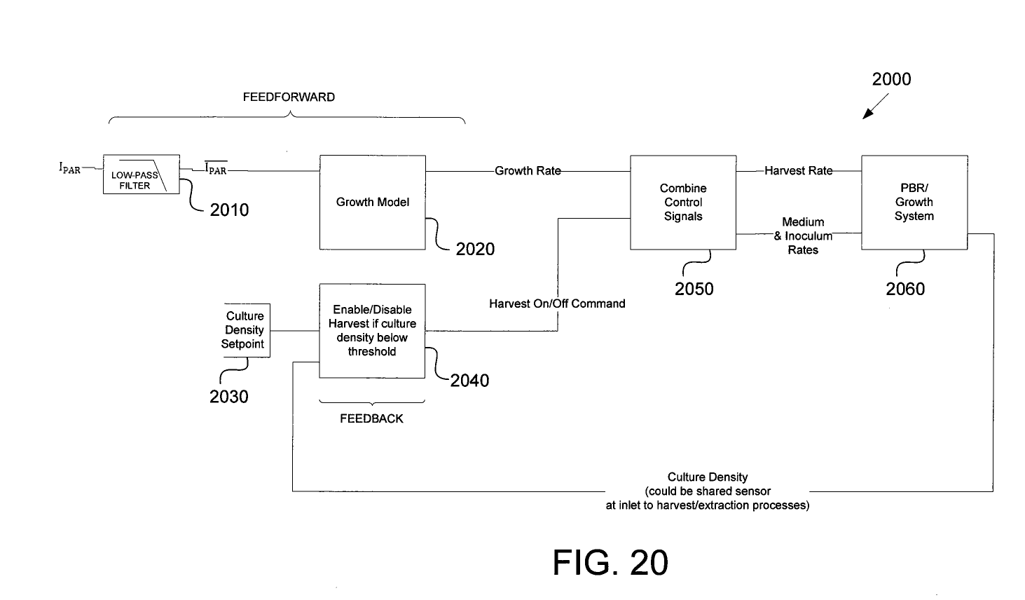

FIG. 20 is a block diagram 2000 illustrating an example of a liquid control system with model-based feedforward components in accordance with one or more embodiments of the present invention. The dynamic model described in the first section is used to determine when to harvest the algae based on microalgae accumulation, according to embodiments of the present invention. In this scenario, the growth model 2020 receives an input PAR that may be filtered using low pass filter 2010. Growth model 2020 uses the PAR value to provide a growth rate to harvest control signal decision block 2050, according to embodiments of the present invention.

In the feedback path, the harvest enable block 2040 is initialized with the starting culture density 2030 and receives dry mass measurements from the actual PBR system 2060. According to various embodiments, the dry mass measurement may be a continuous measurement or an intermittent measurement. The dry mass measurement may also come from every reactor or a shared measurement from one or a few reactors. When the dry mass is above a user specified measurement value, as determined by the harvest enable block 2040, a harvest ON command is sent to the harvest control signal decision block 2050. In this case, algae is harvested from the PBR 2060 at the rate of growth provided by the growth model 2020 and nutrient rich media is sent to the PBR 2060 at the same rate. When the enable harvest 2040 sends an OFF signal to the harvest control signal decision block 2050, no algae is harvested and no nutrient rich media is sent to the PBR 2060.

FIG. 21 illustrates an example of a Labview implementation 2100 of a feedback controller with anti-windup with which some embodiments of the present invention may be utilized. A diagram of a PI controller used for pH control in some embodiments of the present invention is shown in block 2100. In the embodiments illustrated in FIG. 21, there are two memory elements that hold the previously calculated error and control output. In the control (e.g. Labview) software, these memory elements may be shift registers, according to embodiments of the present invention

The dynamic model described herein in the first section could be used to feed an operational research model to determine when to harvest algae, based on expected growth and market conditions, according to embodiments of the present invention. The operational model can take into account the current value of all of the byproducts, the projected values over the next block of time (e.g., a period of days), the projected growth (based on the dynamic model), and the associated operating costs of running the reactor to determine the most profitable time to harvest, according to embodiments of the present invention. Two scenarios are given below:

According to a first scenario, the goal is to harvest algae at the maximum lipid content of the day. It is best to harvest after the microalgae have received 2 moles of photons per m2 for the day provided that the temperature never rose above a certain predetermined temperature (e.g. 25 C). If the temperature does rise above the predetermined temperature, harvest immediately. These conditions will vary with the weather conditions and the time of year and an operational research type model can account for these variations. The actual factors affecting the optimal time may be more complex than the scenario described herein.

According to another scenario: the microalgae is harvested today, it will produce x dollars in lipids and other byproducts. Based on the predicted weather and market conditions over the next couple of days, the yield will be increased to y dollars in lipids and other byproducts but incur z dollars in operational cost. In this scenario, the choice to harvest is based on the scenario that makes the company the most money. Here x and z are can be determined in a relatively straightforward manner, but the accuracy of y depends on both the accuracy of the dynamic model of lipid formation and operational research model of the future market, according to embodiments of the present invention.

### 1.5. Fault Detection

According to some embodiments of the present invention, models that get the same commands as the physical system run in parallel with the actual system. If there are any significant variations between the model output and the measured output, a warning or error may be generated along with an action to take, according to embodiments of the present invention. To illustrate this, a few examples are given.

In a first example, the pH has been constant, and the dissolved O₂ sensor and CO₂ flow rate have been consistent with the model. The model reads that the amount of dry mass is 2 g/L and the optical density (“OD”) sensor reads 20 g/L. In such a scenario, the OD sensor is reading incorrectly and the sensor needs maintenance; the control system may send a message to the operator.

In a second example, the pH is rising and the CO₂ MFC reading is at its maximum. In such a scenario, the control system may send an error message indicating a CO₂ delivery failure, with possible causes including that the CO₂ tank is empty or a hose is not connected, according to embodiments of the present invention.

In a third example, the pH is constant, the amount of CO₂ required is dropping, the dissolved O₂ is dropping, and the OD sensor is decreasing, but the model suggests that growth should be happening. The control system may send an error message indicating that there is something wrong with the algae culture, according to embodiments of the present invention.

### 2. Predictive Control

### 2.1 Benefits

According to some embodiments of the present invention, benefits from using prediction to control a photobioreactor include accounting for process delays and anticipating future conditions. Both of these results improve resource utilization and increase productivity.

Processing delays include delivery delays and transportation delays, according to embodiments of the present invention. The delivery delays include the time that it takes from when a command input is sent to the time it takes for the result of that input to reach the algae. An example of this is the time it takes from when a carbon dioxide flow rate is increased to when the additional carbon dioxide is actually available for the algae to use. Transportation delays include delays from when the conditions change until they are detected by sensors, according to embodiments of the present invention. Continuing with the previous example, after the commanded carbon dioxide flow rate has changed the pH at the point of injection, a period of time will pass before a sensor registers the changed pH. This is a transportation delay. These effects can be significantly minimized by using prediction in photobioreactor control. The benefits from this include improved utilization by applying carbon dioxide as it is needed and not in excess (which may be lost through the vent), according to embodiments of the present invention.

The second benefit according to embodiments of the present invention comes from the ability to predict future conditions. An example of this is illustrated in the following scenario. As the amount of available sunlight changes, so does the algal growth rate. As the growth rate changes, the amount of required carbon dioxide changes too. A prediction of the future sun intensity may be used to promote the correct carbon dioxide concentration when light hits the algae, which will improve algal growth (and hence increase productivity), according to embodiments of the present invention.

### 2.2 Description

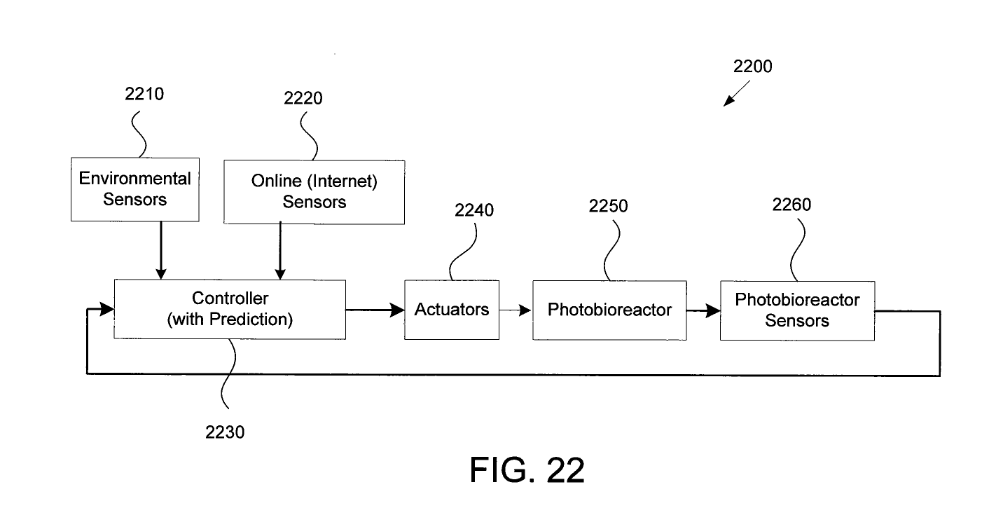

FIG. 22 is a block diagram illustrating an example of a predictive control system 2200 that uses a controller that predicts future events to compute control actions in accordance with some embodiments of the present invention. The general architecture with a predictive controller 2200 consists of sensor readings 2210 and 2220, and a predictive controller 2230 that feeds actuators 2240 in a PBR 2250. Sensors from the PBR 2260 feed signals back to the predictive controller 2230.

Examples of actuators that may be used in some embodiments of the present invention are ON-OFF valves that may be controlled either manually or electronically, mass flow controllers that supply a (controller-) specified amount of gas, or a peristaltic pump that delivers or removes liquids at a given rate.

Environmental sensors 2210 provide measurements from sensors that are located at the algae growth system site and online sensors 2220 provide measurements from the Internet, according to embodiments of the present invention. Both types of sensors provide information about the environmental surroundings. This information may be used to determine the algae metabolic activity. In accordance with various embodiments, these measurements may include the sun intensity, cloud cover, air temperature, and humidity. The sun intensity is a significant measurement, which may be measured as total radiation, direct and diffuse radiation, and photosynthetically active radiation (PAR). All of these radiation measurements are a function of sun position, cloud cover, and the PBR orientation, according to embodiments of the present invention. The sun position is a function of the day of year, the time of day, and the physical location of the algae growth system (AGS) (e.g., the longitude and latitude of the AGS). Online Sensors 2220 may also provide their own prediction about future environmental conditions. In some embodiments, some or all of these predictions may be generated along with a “confidence parameter” that the predictor can use to select its control action.

PBR Sensors 2260 are sensors that provide any measurements with information about the current state of the algae and surrounding media in the AGS. These measurements may include pH, dissolved carbon dioxide (aqueous), total dissolved carbon (TDC), dissolved oxygen, output (vent) carbon dioxide gas, output (vent) oxygen gas, temperature, pressure, flow rate, dry mass, optical density, cell count, chlorophyll mass, available nutrients, and/or PBR working volume, according to embodiments of the present invention. These measurements may be obtained from automated devices or manual measurements, according to embodiments of the present invention. Measurements may be frequent (e.g., multiple measurements per second) to less frequent (one a day to once a week), and may also be taken more frequently or less frequently than the examples given, according to embodiments of the present invention.

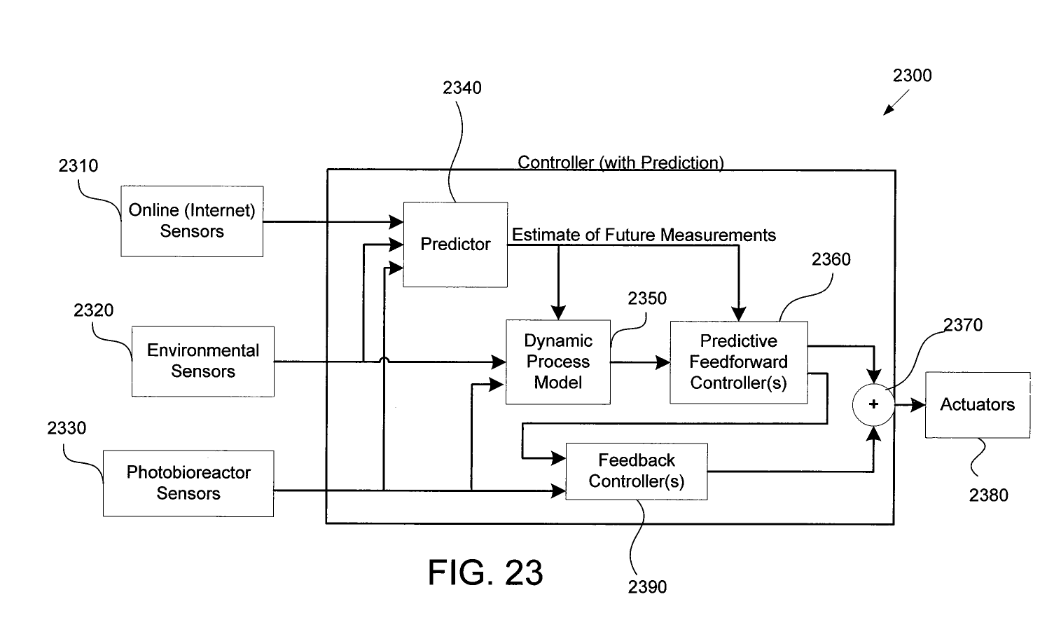

FIG. 23 is a block diagram illustrating an example of a predictive control system in accordance with one or more embodiments of the present invention. Predictor module 2340 uses some or all of the currently available measurements 2310, 2320, and 2330 (e.g., the current measurements and all previous measurements) to estimate future sensor measurements, according to embodiments of the present invention. When online sensors 2310 are providing their own predictions of future events, the predicted information may be used by the predictor module 2340 to improve the “Estimates of Future Measurements.” Examples of predicted events include, but are not limited to, future sun intensity, cloud cover, and temperature. The “Estimates of Future Measurements” may be passed to the dynamic process model 2350 and predictive feedforward controller(s) 2360, which use predicted measurements to choose the best control action to maximize future performance, according to embodiments of the present invention. According to some embodiments, predictor module 2340 and/or online sensors 2310 may be referred to as a prediction module because they can be configured to estimate a set of future environmental conditions over a future time period.

In the architecture described above, predictor module 2340 may pass values directly to the feedback controller 2390 by adding a direct feedthrough term to the predictive feedforward controller(s) 2360. The outputs of the feedforward and feedback controllers can be summed using summer 2370. The resulting signal can then be sent to the actuators 2380.

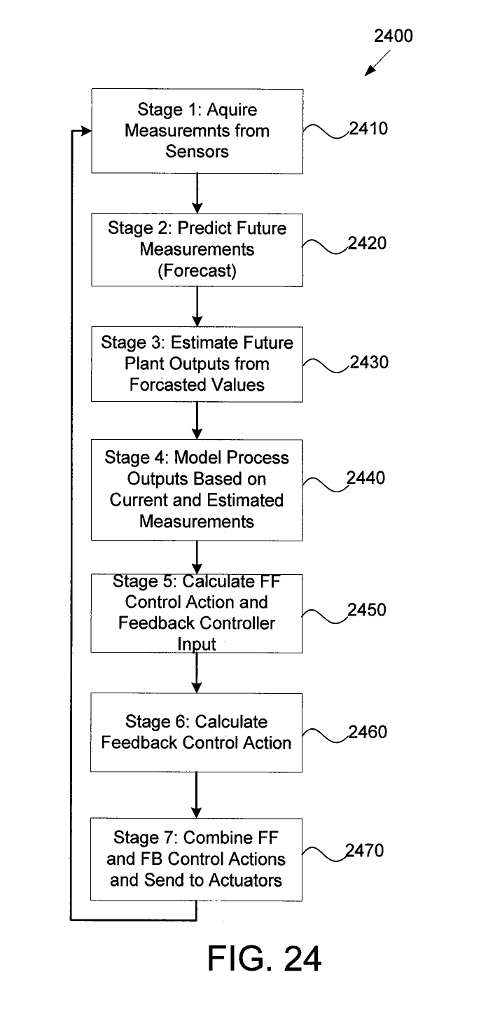

FIG. 24 is a flowchart 2400 illustrating a set of exemplary operations used to implement a predictive control strategy in accordance with various embodiments of the present invention. According to some embodiments of the present invention, Stage 1 includes acquisition operation 2410 to acquire measurements. Acquisition operation 2410 may be done, for example, by sensors 2310, 2320, and 2330. Stage 2 includes a forecasting operation 2420 to predict future measurements. Stage 3 includes an estimation operation 2430 to estimate future plant outputs from the forecasted values. In some embodiments, stages 2 and 3 may be performed by the predictor module 2340 in FIG. 23. In some embodiments, measurements can be predicted by the online sensors 2310. In such cases, forecasting operation 2420 may be skipped.

Stage 4 includes modeling operation 2440 to model process outputs based on current and estimated measurements. In some embodiments, modeling operation 2440 may be done by the dynamic process model 2350 in FIG. 23. Stage 5 includes calculation operation 2450 to calculate the FF control action and feedback controller input. In some embodiments, calculation operation 2450 may be done by the predictive feedforward controller(s) 2360. Stage 6 includes control calculation operation 2460 to calculate the feedback control action. Operation 2460 may be done by the feedback controller(s) 2390, according to embodiments of the present invention. Once operation 2460 is complete, combination operation 2470 is done. In some embodiments, operation 2470 may be performed by the summer 2370 and actuators 2380.

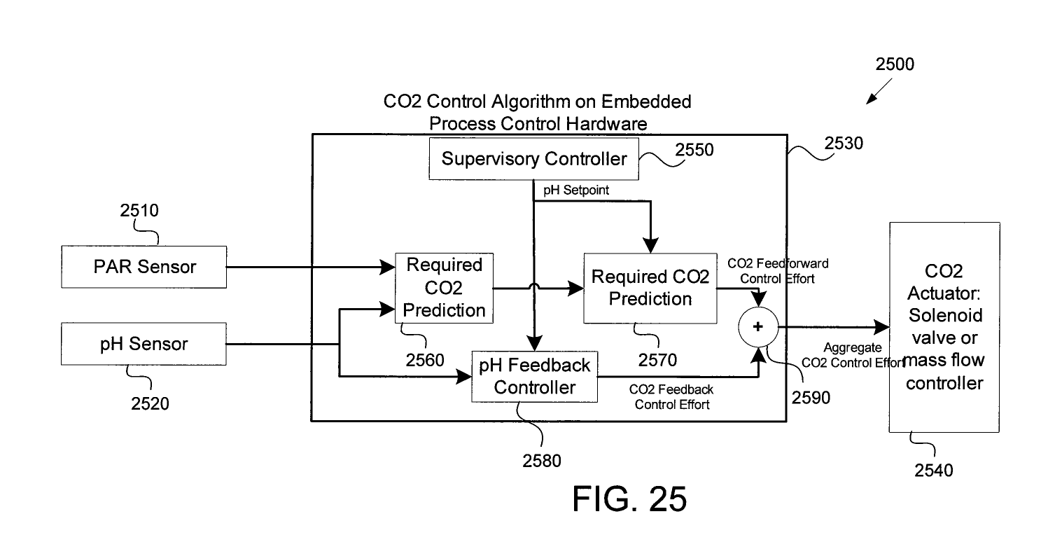

FIG. 25 is a block diagram 2500 illustrating an example architecture for a predictive control system in accordance with one or more embodiments of the present invention. PAR can be measured by PAR sensor 2510 and pH can be measured by pH sensor 2520. In the embodiments illustrated in FIG. 25, the measurement values are passed to the CO₂ forecast prediction feedforward controller 2560. The measured pH can be sent to the feedback controller 2580. Supervisory controller 2550 sends information about the pH set point to both the feedforward controller 2570 and feedback controller 2580 that will be used to determine feedforward and feedback CO₂ flow rates. Summer 2590 sums the flow rates and then communicates them to the CO₂ actuators 2540, according to embodiments of the present invention.

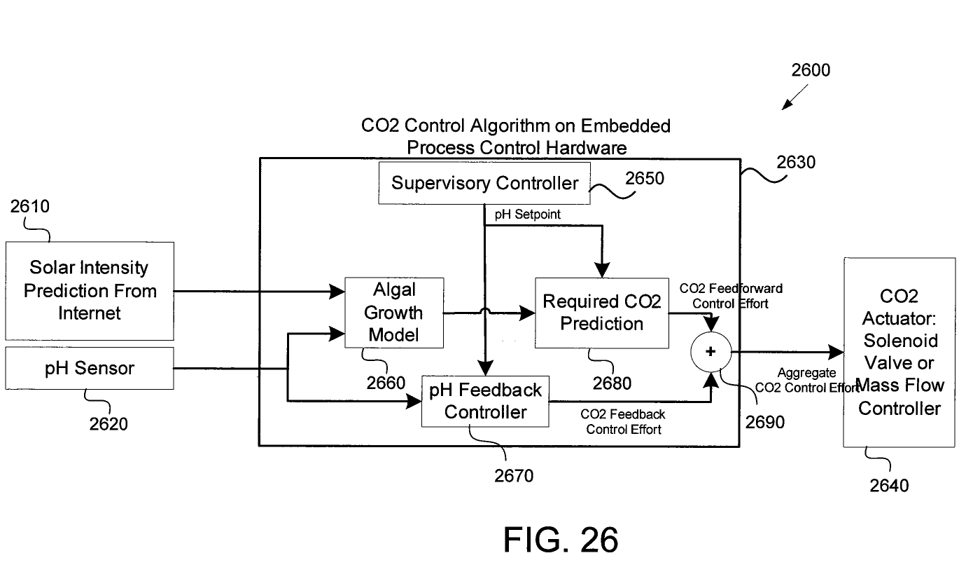

FIG. 26 is a block diagram 2600 illustrating an example architecture for a predictive control system with predictive pH regulation using the growth model and pH feedback in accordance with one or more embodiments of the present invention. In particular, FIG. 26 is a hardware diagram illustrating a block diagram of a system for regulating pH using a combination of feedforward and feedback controllers via CO₂ injection based on measured and predicted PAR. The measured and predicted PAR 2610 and measured pH 2620 are passed to the feedforward controller 2660. The measured pH 2620 is sent to the feedback controller 2670. Based on predicted PAR 2610, a CO₂ prediction is calculated by the algal growth model 2660. A supervisory controller 2650 sends information about the pH set point to both the feedforward controller 2680 and feedback controller 2670 that will be used to determine feedforward and feedback CO₂ flow rates, according to embodiments of the present invention. The flow rates are summed using summer 2690 and sent to the CO₂ actuators 2640.

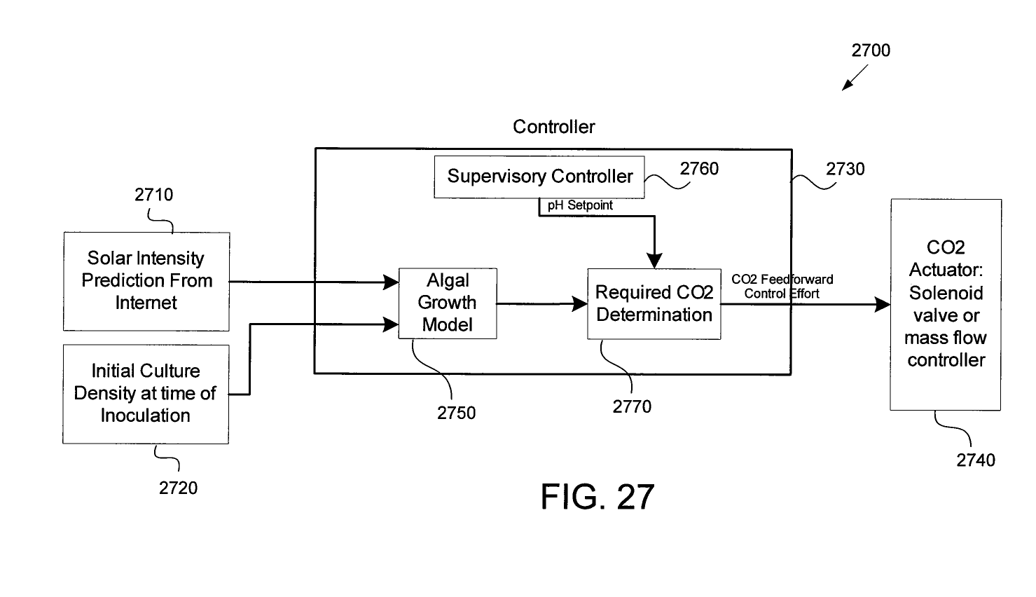

FIG. 27 illustrates a block diagram 2700 showing with an exemplary set of components for the implementation of a controller using open loop predictive pH regulation using a growth model in accordance with various embodiments of the present invention. In the particular embodiments shown in FIG. 27, block diagram 2700 illustrates a hardware diagram for regulating pH using only feedforward controllers via CO₂ injection based on measured and predicted PAR. The measured and predicted PAR 2710 and initial dry mass at inoculation 2720 are passed to the feedforward controller 2750. Based on predicted PAR 2710, a CO₂ prediction is calculated by the algal growth model 2750. A supervisory controller 2760 sends information about the pH set point to the feedforward controller 2770 that will be used to determine the commanded CO₂ flow rate. The flow rate is sent to the CO₂ actuators 2740.

### 3. Adaptive Control

### 3.1 Benefits

According to some embodiments of the present invention, adaption and learning are used to improve performance by customizing and/or adapting the model parameters to the actual plant that is being controlled. Adaptive learning may also be used to track plant dynamics as they change over time due to “wear and tear.” This can be used to maintain plant performance as the plant changes over time, according to embodiments of the present invention.

### 3.2 Description

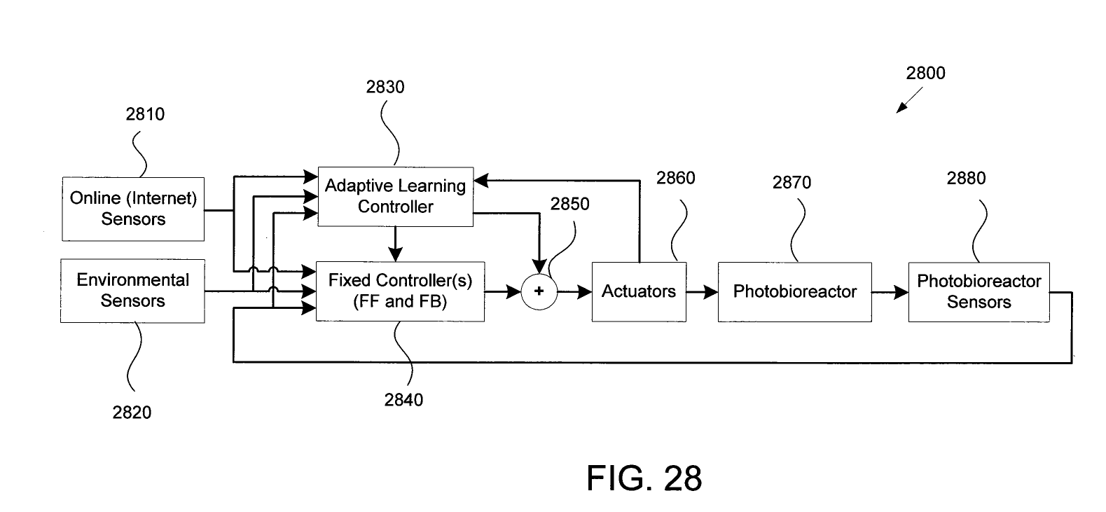

FIG. 28 is a block diagram 2800 illustrating an example architecture for an adaptive control system in accordance with one or more embodiments of the present invention. In the control strategy depicted in FIG. 28, the fixed controller 2840 can be a fixed structure (e.g., a PI controller) which may have controller parameters that are adapted over time, according to embodiments of the present invention. However, this adaptation may be used in some circumstances and not others. According to some embodiments of the present invention, the adaptive learning controller 2830 uses experience with the physical system 2870 to optimize the performance of the controllers.

Online sensors 2810 and environmental sensors 2820 are used along with PBR sensors 2880 to feed the adaptive learning controller 2830 and fixed controller(s) 2840. The outputs from the two controllers 2830 and 2840 can be summed using summer 2850 and sent to the actuators 2860, which are experienced on the PBR 2870.

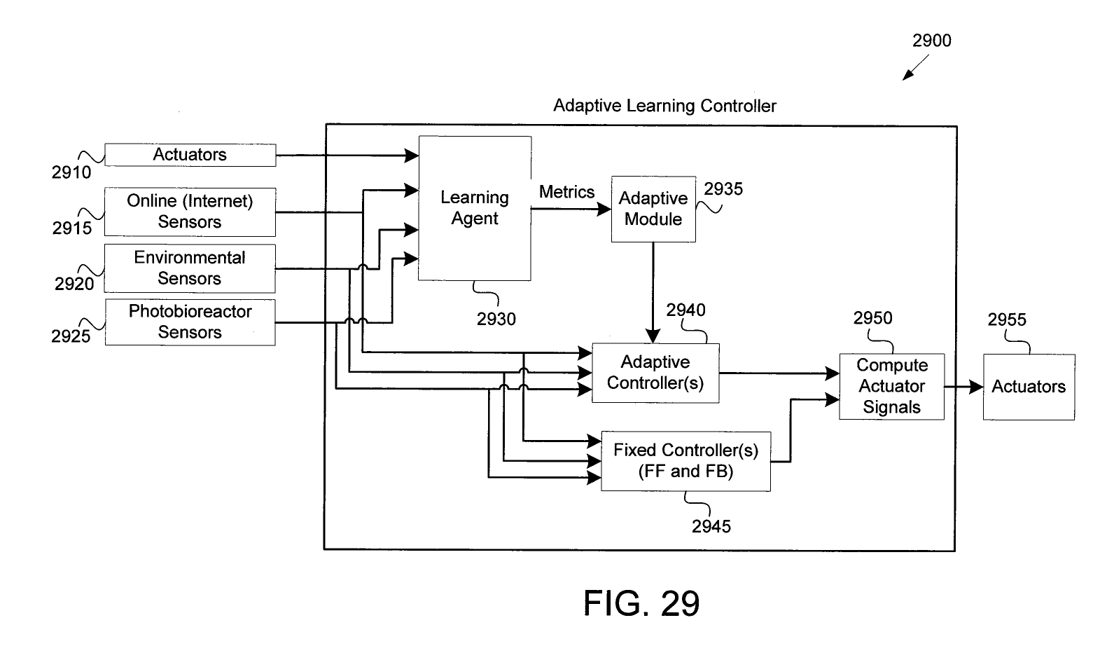

FIG. 29 is a block diagram 2900 illustrating an example architecture for an adaptive learning control system in accordance with one or more embodiments of the present invention. According to some embodiments of the present invention, the learning agent 2930 looks at the control actions sent to the actuators 2910 and resulting measurements from the online sensors 2915, environmental sensors 2920, and PBR sensors 2925. Based on the desired outputs, performance metrics can be calculated using learning agent module 2930. These metrics are generally a measure of how close the observed outputs are to ideal outputs, but could be any metric that improves performance (e.g., they could be penalties on large control signals or high frequency control). They are used to both determine the controller update and/or to improve a model of how well the control actions meet a performance objective in the adaptive module 2935. In the first case, the metric can be used to directly affect specific controller parameters. Examples of this include, but are not limited to, gradient decent and line searches. In the second case, the metric may be used to determine the effectiveness of a control action. One example of this is reinforcement learning.

In reinforcement learning, reinforcement signals are calculated based on the current controller actions and plant observations. A reinforcement learner keeps track of all of the observed reinforcement signals and (over time) determines the best control action to maximize future rewards (or minimize future penalties). Because this is a continuing process, the reinforcement learner is able to adapt to different conditions as they occur, according to embodiments of the present invention. This ability to adapt comes from a “forgetting factor” that allows the reinforcement learner to decide the best control actions based on more current events and less on older events, according to embodiments of the present invention.

Based on the parameter update calculated by the adaptive module 2935, the adaptive controller 2940 is updated in some embodiments of the present invention. In the case of an actor-critic reinforcement learning algorithm, the adaptive module 2935 may be the critic and the adaptive controller 2940 may be the actor. In addition to updating the adaptive controller 2940, the adaptive module 2935 may be used to update parameters in the fixed controller 2945, according to embodiments of the present invention. The outputs of the adaptive controllers 2940 and fixed controllers 2945 are used to compute the actuator signals 2950, which are sent to the actuators 2955. The computation of the actuator signals 2950 may be an addition of the two signals, a weight average based on “quality” of the data (e.g., as done in a Kalman filter), or another mapping that produces a desired output, according to embodiments of the present invention.

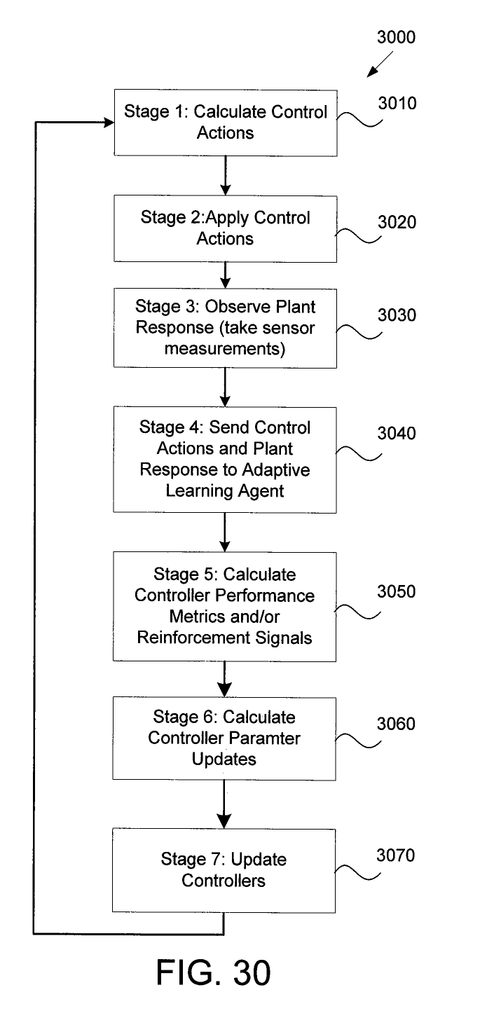

FIG. 30 is a flowchart 3000 illustrating an exemplary set of operations for the operation of an adaptive control system that may be used with various embodiments of the present invention. Calculation operation 3010 calculates one or more control actions. In some embodiments, calculation operation 3010 may be done by the combining the fixed controller outputs 2860 (see FIG. 28) with current adaptive controller outputs 2840 (e.g., the output when no adaptation has taken place). Application operation 3010 applies the calculated control actions calculated to the PBR (e.g., 2870 in FIG. 28), according to embodiments of the present invention. Measurement operation 3030 reads in the resulting plant response from the control actions in Stage 2, according to embodiments of the present invention. In Stage 4, communication operation 3040, sends the control actions and the plant response to the adaptive learning agent. For example, the previous plant inputs and outputs may be sent to the learning agent 2930 in FIG. 29, according to embodiments of the present invention. In Stage 5, calculation operation 3050 can calculate performance metrics and send them to the adaptive module, according to embodiments of the present invention. Stage 6 uses update operation 3060 to determine how the controllers should be updated. Then in Stage 7, configuration operation 3070 updates the adaptive controller(s) (e.g., 2940 in FIG. 29) and possible parameters in the fixed controller(s) (e.g., 2945 in FIG. 29), according to embodiments of the present invention. According to some embodiments, the process 3000 repeats.

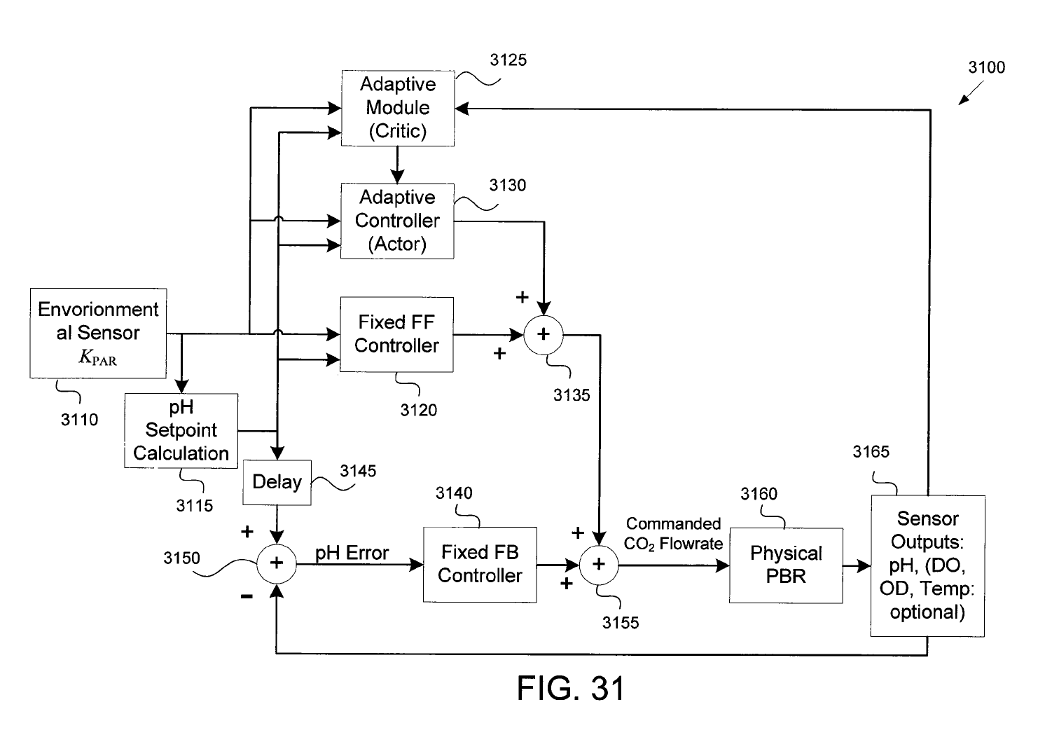

FIG. 31 is a block diagram 3100 illustrating an exemplary set of components for implementing a controller with adaptive feedforward control along with feedback pH regulation with feedforward dead-time compensation in accordance with some embodiments of the present invention. The embodiments illustrated in FIG. 31 use environmental sensors are used to measure the incident light I_{PAR} 3110. Based on the current light intensity, a target pH is chosen and set in setpoint module 3115. The desired pH and measured incident light I_{PAR} are sent to adaptive module 3125, adaptive controller 3130, and fixed feedforward controller 3120. The feedforward controllers, namely the actor 3130 and fixed controller 3120, each calculate their feedforward control signals, which are summed together using summer 3135 to form the feedforward control signal.

In the feedback path, the desired pH from setpoint module 3115 is sent to the delay module 3145 that accounts for the transport delay between when the feedforward CO2 flowrate control signal is received at the PBR 3160 and when the pH sensor 3165 will react to the addition of more CO₂. In the feedback path the measured pH 3165 is subtracted from the delayed pH setpoint 3145 and sent to the fixed feedback controller 3140. The results of the feedback 3140 and feedforward 3135 are summed at 3155 and sent to the actuators on the physical PBR 3160. After the commanded CO₂ has been delivered to the physical PBR 3160, the sensors 3165 are output to the adaptive module (critic) 3125. The adaptive module 3125 receives the PBR sensor measurements 3165, environment sensor measurements 3110, and pH setpoint 3115, and calculates a correction (or innovation) that is sent to the adaptive controller (actor) 3130. The actor 3125 receives the signal from the critic 3125 and updates the adaptive controller 3130.

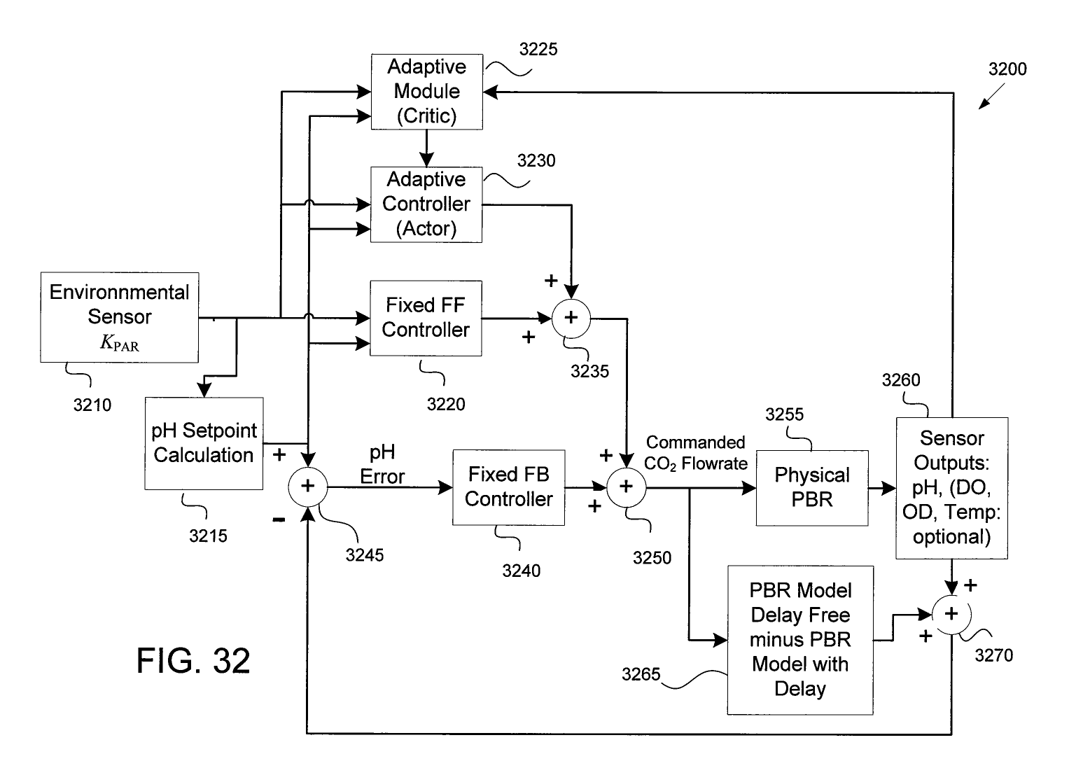

FIG. 32 is a block diagram 3200 illustrating an exemplary set of components for implementing a controller with adaptive feedforward control along with feedback pH regulation with Smith predictor dead-time compensation in accordance with one or more embodiments of the present invention. This is a specific way to implement an adaptive controller with the intent to improve performance (i.e., increase biomass production and reduce CO2 losses). This method uses a Smith predictor for dead time compensation.

In the embodiments illustrated in FIG. 32, environmental sensors are used to measure the incident light I_{PAR} 3210. Based on the current light intensity, a target pH is chosen 3215. The desired pH and measured incident light I_{PAR} are sent to adaptive module 3225, adaptive controller 3230, and fixed feedforward controller 3220. The feedforward controllers, namely the actor 3230 and fixed controller 3220, each calculate their feedforward control signals, which are summed together at 3235 to form the feedforward control signal. In the feedback path, the measured pH 3260 is modified, based on model of the plant 3265, so that transport delay between when the feedforward CO₂ flowrate control signal is received at the PBR 3355 does not appear in the calculated pH error 3240. This is done using a standard Smith Predictor (i.e., the combination of 3255, 3260, 3265, and 3270). The delay free pH measurement 3270 is subtracted 3245 from the pH setpoint 3215. The calculated pH error is received by the fixed feedback controller 3240.

The results of the feedback 3240 and feedforward 3235 are summed 3355 and sent to the actuators on the physical PBR 3255. After the commanded CO₂ has been delivered to the physical PBR 3255, the sensors 3260 are output to the adaptive module (critic) 3225. The adaptive module 3225 receives the PBR sensor measurements 3260, environment sensor measurements 3210, and pH setpoint 3215, and calculates a correction (or innovation) that is sent to the adaptive controller (actor) 3230. The actor 3225 receives the signal from the critic 3225 and updates the adaptive controller 3230.

### 4. Additional Embodiments: Model-Based Control Using Feedforward

### 4.1 Benefits

Accuracy of control is improved and cost of sensors is reduced by use of a dynamic model of system behavior that is used to predict parameters that are not directly sensed but are, instead, estimated from modeled system dynamics based on sensed parameters or based on readily available weather information, according to embodiments of the present invention.

### 4.2 Description

A generic diagram of a system that employs a feedforward control strategy is shown in FIG. 8. The controller is the component that performs calculations relevant to the control strategy, according to embodiments of the present invention. Environmental sensors provide information about external parameters (e.g. incident sunlight intensity) to the controller. Actuators transduce the controller actuation requests into physical parameters that are imposed upon the photobioreactor. The photobioreactor sensors transduce selected physical parameters relevant to the control strategy into signals that can be detected by the controller, according to embodiments of the present invention.

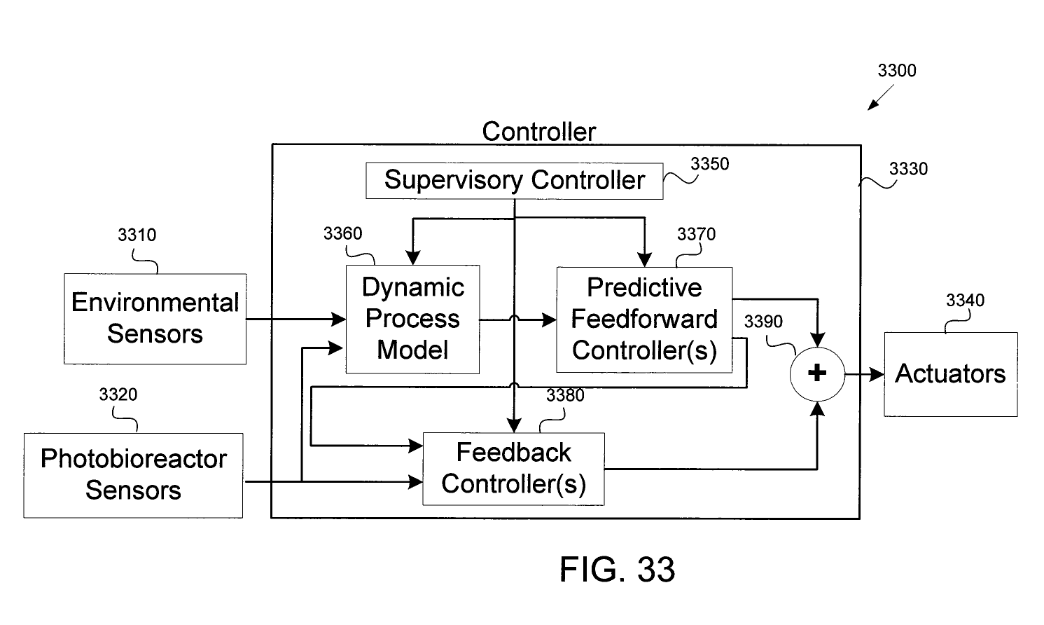

FIG. 33 illustrates a block diagram showing a predictive control system that may be used with some embodiments of the present invention. The embodiments illustrated in FIG. 33 depict the internal topology of the controller algorithms. A supervisory controller 3350 determines the desired operating mode of the dynamic process model 3360, the predictive feedforward controller 3370, and the feedback controller 3380. The dynamic process model 3360 employs signals from the environmental sensors 3310 and photobioreactors 3320 to simulate the relevant dynamics of the processes occurring within the photobioreactor that affect the parameters to be controlled. Relevant dynamics include light delivery to the active culture, gas transfer, algal photosynthesis, algal metabolism and nutrient uptake, algal culture hydrochemistry, and/or thermal behavior, according to embodiments of the present invention. The predictive feedforward controllers 3370 determine desired actuator behavior based on estimates of system parameters delivered by the dynamic process model 3360, according to embodiments of the present invention. The feedback controllers 3380 employ signals from the photobioreactor sensors 3320 in order to determine dynamically adapted actuator commands to correct differences between actual and desired parameters that are both controlled and directly sensed. The outputs from the predictive feedforward controller(s) 3370 and the feedback controller(s) 3380 are summed 3390 and then sent to the actuators 3340, according to embodiments of the present invention.

Some detailed calculations relating to the feedforward control and growth model are shown with respect to FIG. 8. A sample flowchart for this process is shown in FIG. 9.

According to embodiments of the present invention, the growth model predicts the amount of algal dry mass (malgae) based on input sun intensity (uPAR) and a few system parameters, namely a sun utilization constant (KPAR) and a rate of respiration in the dark constant (R). The state space equations for the open loop feedforward growth model are

\dot{m}_{algae}=(K_{PAR}u_{PAR}−R)m_{algae}=uₕₐᵣᵥₑₛₜ

y_{DM}=m_{algae}

Here, uₕₐᵣᵥₑₛₜ is the commanded harvest rate for removing algae from the PBR, according to embodiments of the present invention. This could be used in a continuous reactor to maintain a specific algal density. In a batch reactor, this term would be zero. The term y_{DM}=m_{algae} is used explicitly point out that the model output is the algae dry mass. A block diagram of an open loop model is shown in FIG. 17.

Running the model in an open loop scenario can cause the outputs to drift in some cases. In cases where a measurement of the dry mass is available, an observer may be used in some embodiments to correct for the model inaccuracies. To create an observer from the previous model, a correction term is added to the differential equation, according to embodiments of the present invention. The modeled dry mass state is \hat{m}_{algae} its derivative is {circumflex over (\dot{m}_{algae}, and the modeled dry mass output is ŷ_{DM}. Now, y_{DM} is the actual (measured) dry mass that comes from either an automated instrument or an operator (e.g., a lab technician), according to embodiments of the present invention. The state space equations for an observer based feedforward model according to embodiments of the present invention are given by

{circumflex over (\dot{m}_{algae}=(K_{PAR}u_{PAR}−R)\hat{m}_{algae}−uₕₐᵣᵥₑₛₜ+L(ŷ_{DM}−y_{DM})

ŷ_{DM}=\hat{m}_{algae}

Based on the observed dry mass growth rate ({circumflex over (\dot{m}_{algae}), the feedforward carbon dioxide flowrate \dot{m}_{CO}_{₂} may be determined. In some cases, extra signal processing may be done on {circumflex over (\dot{m}_{algae} to smooth or filter the signal. The FF carbon dioxide rate is then given by

\dot{m}_{CO}_{₂}=K_{CO}_{₂}{circumflex over (\dot{m}_{algae}

FIG. 20 shows a block diagram that illustrates the following:

The actual computation of {circumflex over (\dot{m}_{algae} may be done by numerical integration, which may be accomplished in many different ways, according to embodiments of the present invention.

System diagrams of embodiments of the present invention are shown in FIG. 4. According to some embodiments of the present invention, a feedforward-plus-feedback configuration uses on-site sensors:

According to other embodiments of the present invention, a feedforward plus feedback configuration is used in which the environmental information (e.g. sunlight intensity) is inferred not from an on-site sensor but instead from publicly available current weather information available via the internet. This is illustrated in FIG. 26, for example.

Finally, according to yet other embodiments of the present invention, a pure feedforward configuration employs publicly available weather condition data and includes no real-time feedback sensor. In this embodiment, only the initial culture density and externally provided data are used in order to maintain an effective level of carbon dioxide delivery. This is illustrated in FIG. 27, for example.

### 5. Fault Detection

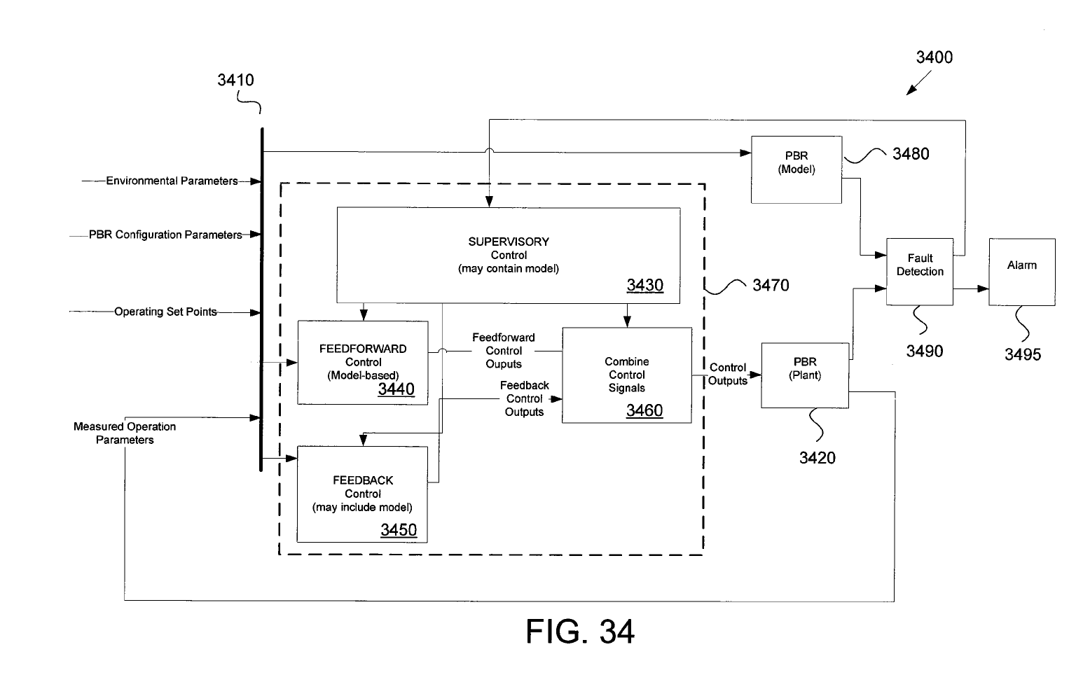

FIG. 34 illustrates an example of a fault detection based supervisory control system 3400 according to one or more embodiments of the present invention. The model illustrated in FIG. 34 is similar to the model of FIG. 4 with an additional fault detection component. Based on detected faults, the supervisory controller alters the control signals to maintain proper system operation. According to some embodiments of the present invention, the controller inputs are read in at interface 3410. One or more of the controllers described above, or one known to those of ordinary skill in the art, can be used for feedforward 3440 and feedback 3450 controllers. The controllers are implemented in block 3470 and the actuator control signal 3460 is sent to the actual plant 3420, according to embodiments of the present invention.

In parallel, the inputs 3410 are sent to the PBR model 3480 according to various embodiments. The outputs from the model 3480 and actual plant 3420 can be compared 3490 to see if there is a fault. In some embodiments, if there is a fault, an alarm 3495 is triggered and a fault is sent to the supervisory controller 3430. When there is a fault, the supervisory controller 3430 will adjust the actuator control signal 3460 to maintain proper operation and will send signals to the feedforward 3440 and feedback 3450 controllers to signify that some (or all) of the measurement signals are corrupt and should not be used to update the controller. An example of this would be if the pH sensor fails, then the feedback controller 3450 that is regulating pH should ignore the apparently large error between the setpoint and measured pH when trying to calculate its CO2 flowrate control signal. Once the fault has been cleared in some embodiments, feedback 3450 controller is reset by the supervisory controller 3430 and the controller block 3470 operates normally as if there were no fault.

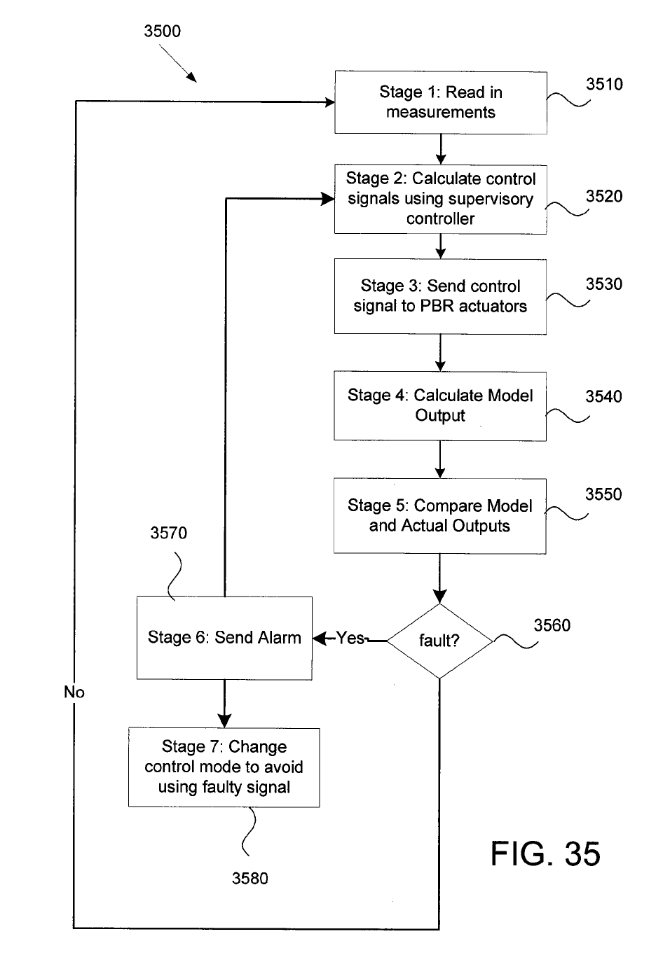

FIG. 35 is a flowchart 3500 illustrating an exemplary set of operations that may be used for fault detection based supervisory control in accordance with various embodiments of the present invention. In stage 1 3510, measurements are read in from the environmental and PBR sensors. Stage 2 3520 calculates the controller actuation signals. If there is a fault, the supervisory controller overrides the feedforward and feedback controllers. If there is no fault, the feedforward and feedback controllers operate normally. In stage 3 3530, the computed controller actuation signal is sent the actual PBR. In Stage 4 3540, the model outputs are calculated. In Stage 5 3550, the model and actual outputs are compared and a fault signal is generated. For instance, if |actual value−measured value|>user defined threshold, then a fault or error is triggered. If there is a fault, an alarm 3570 is generated in Stage 6 and/or sent to the supervisory controller in Stage 2 3520. In stage 7 3538, the supervisory controller sends signal(s) to the control modules(s) to ensure that the suspect signals are not used by the feedforward and feedback controllers. If there are no faults, the process returns to Stage 1 3510 and repeats.

### 6. Implementations

The models and methods presented herein have been described in terms of both differential (continuous time) and difference (discrete time) equations. Differential equations may be implemented using either analog circuit components (e.g., resistors, capacitors, inductors, operational amplifiers, transconductance/transresistance amplifiers, etc.) or through numerical integration (e.g., forwards or backwards Euler/1st order Runge-Kutta, 2nd/3rd/4th/higher order Runge-Kutta, Domand-Prince (ode45), Bogacki-Shampine (ode23), Adams (ode113), stiff/NDF (ode15s), stiff/Modified Rosenbrock (ode23s), Mod. Stiff/Trapezoidal(ode23t), stiff/TR-BDF2 (ode23tb)). Difference equations are also implemented numerically. Numerical methods may be implemented on either dedicated hardware (e.g., PLC, FPGA, DSP chip, compact Fieldpoint, Fieldpoint, Compact Rio, and any other generic DAQ device that can implement numerical algorithms locally) or on larger systems (e.g., host computer, mainframe, server, etc.) that are located on-site or communicate wirelessly (e.g., via the Internet) to a system off site.

Embodiments of the present invention may be provided as a computer program product that may include a machine-readable medium having stored thereon instructions that may be used to program a computer (or other electronic devices) to perform a process. The machine-readable medium may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments of the present invention may also be downloaded as a computer program product, wherein the program may be transferred from a remote computer to a requesting computer by way of data signals embodied in a carrier wave or other propagation medium via a communication link (e.g., a modem or network connection).

For the sake of illustration, various embodiments of the present invention have herein been described in the context of computer programs, physical components, and logical interactions within modern computer networks. Importantly, while these embodiments describe various aspects of embodiments of the invention in relation to modern computer networks and programs, the method and apparatus described herein are equally applicable to other systems, devices, and networks as one skilled in the art will appreciate. As such, the illustrated applications of the embodiments of the present invention are not meant to be limiting, but instead exemplary.

### Exemplary Computer System Overview

Some embodiments of the present invention include various steps, some of which may be performed by hardware components or may be embodied in machine-executable instructions. These machine-executable instructions may be used to cause a general-purpose or a special-purpose processor programmed with the instructions to perform the steps. Alternatively, the steps may be performed by a combination of hardware, software, and/or firmware. In addition, some embodiments of the present invention may be performed or implemented, at least in part (e.g., one or more modules), on one or more computer systems, mainframes (e.g., IBM mainframes such as the IMB zSeries, Unisys ClearPath Mainframes, HP Integrity NonStop servers, NEC Express series, and others), or client-server type systems. In addition, specific hardware aspects of embodiments of the present invention may incorporate one or more of these systems, or portions thereof.

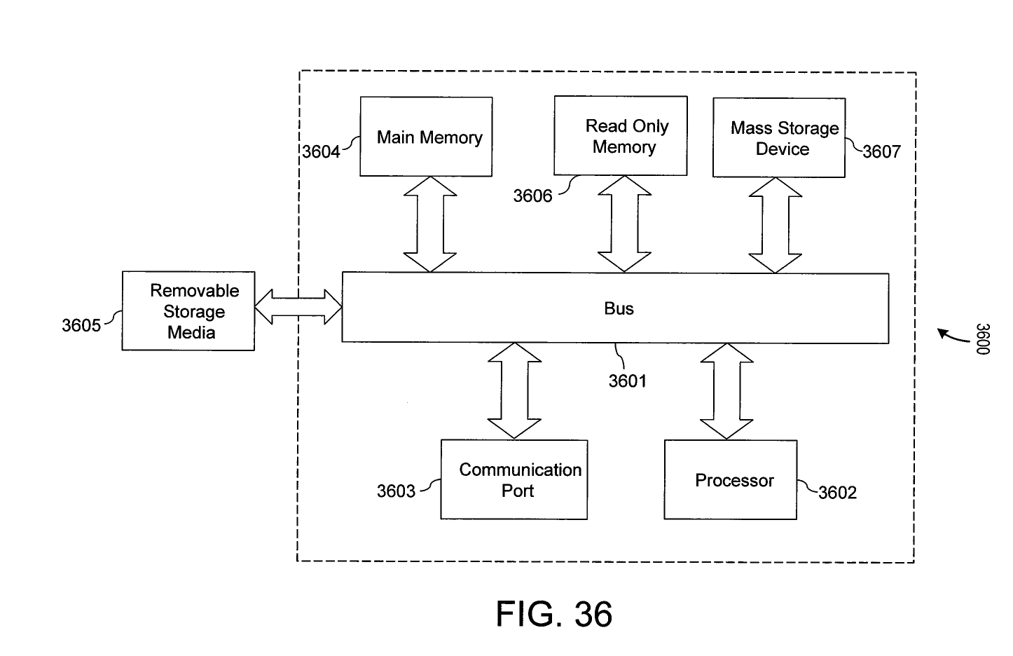

As such, FIG. 36 is an example of a computer system 3600 with which embodiments of the present invention may be utilized. According to the present example, the computer system includes a bus 3601, at least one processor 3602, at least one communication port 3603, a main memory 3604, a removable storage media 3605, a read only memory 3606, and a mass storage 3607.

Processor(s) 3602 can be any known processor, such as, but not limited to, an Intel® Itanium® or Itanium 2® processor(s), or AMD® Opteron® or Athlon MP® processor(s), or Motorola® lines of processors. Communication port(s) 3603 can be any of an RS-232 port for use with a modem based dialup connection, a 10/100 Ethernet port, or a Gigabit port using copper or fiber. Communication port(s) 3603 may be chosen depending on a network such a Local Area Network (LAN), Wide Area Network (WAN), or any network to which the computer system 3600 connects.

Main memory 3604 can be Random Access Memory (RAM), or any other dynamic storage device(s) commonly known in the art. Read only memory 3606 can be any static storage device(s) such as Programmable Read Only Memory (PROM) chips for storing static information such as instructions for processor 3602.

Mass storage 3607 can be used to store information and instructions. For example, hard disks such as the Adaptec® family of SCSI drives, an optical disc, an array of disks such as RAID, such as the Adaptec family of RAID drives, or any other mass storage devices may be used.

Bus 3601 communicatively couples processor(s) 3602 with the other memory, storage and communication blocks. Bus 3601 can be a PCI/PCI-X or SCSI based system bus depending on the storage devices used.

Removable storage media 3605 can be any kind of external hard-drives, floppy drives, flash drives, IOMEGA® Zip Drives, Compact Disc-Read Only Memory (CD-ROM), Compact Disc-Re-Writable (CD-RW), Digital Video Disk-Read Only Memory (DVD-ROM).

The components described above are meant to exemplify some types of possibilities. In no way should the aforementioned examples limit the scope of the invention, as they are only exemplary embodiments.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A system for growing algae comprising:
a photobioreactor containing media, wherein the photobioreactor is subject to one or more environmental conditions, and wherein one or more operation parameters can be adjusted to affect growth of algae in the media;
a modeling unit comprising an algal growth model, the algal growth model relating growth and constituents of the algae in the media to the one or more environmental conditions and the one or more operation parameters;
a control unit configured to access the modeling unit and determine the one or more operation parameters based on the algal growth model and to generate a control signal indicating the one or more operation parameters; and
an actuator unit configured to receive the control signal and adjust the one or more operation parameters based on the control signal.

2. The system of claim 0, wherein the one or more operation parameters are selected to correspond to maximum algal growth according to the algal growth model, given the one or more environmental conditions.

3. The system of claim 0, wherein the one or more environmental conditions comprise light and media pH, wherein the one or more operation parameters comprise carbon delivery rate to the photobioreactor, and wherein the modeling unit generates the control signal according to a timing schedule for carbon delivery.

4. The system of claim 0, wherein the actuator unit is a valve in fluid communication with a supply of carbon dioxide, and wherein the timing schedule for the carbon delivery comprises an intermittent delivery of the carbon dioxide.

5. The system of claim 0, wherein the carbon delivery rate is selected to correspond to maximum algal growth according to the algal growth model, given the light and media pH conditions.

6. The system of claim 0, wherein the one or more environmental conditions comprise light, temperature, algal culture density, and media pH, and wherein the one or more operation parameters comprise carbon delivery rate, media flow rate, and harvesting rate.

7. The system of claim 0, wherein the carbon delivery rate, media flow rate, and harvesting rate are selected to correspond to maximum algal growth according to the algal growth model, given the light and media pH conditions.

8. The system of claim 0, further comprising one or more sensors configured to detect the one or more environmental conditions.

9. The system of claim 0, wherein the control signal is a first control signal, the system further comprising:
a sensor configured to detect a sensed condition of the one or more environmental conditions and generate a sensing signal indicating the sensed condition; and
a feedback control unit configured to receive the sensing signal, compare the sensed condition with a setpoint condition, and generate a second control signal based on the comparison;
wherein the actuator unit is further configured to receive the second control signal and adjust the one or more operation parameters based on the second control signal.

10. The system of claim 0, wherein the control signal is a first control signal, the system further comprising:
a sensor configured to detect a sensed condition of the one or more environmental conditions and generate a sensing signal indicating the sensed condition; and
a feedback control unit configured to receive the sensing signal, compare the sensed condition with a setpoint condition, and generate a second control signal based on the comparison and based on the first control signal;
wherein the actuator unit is further configured to receive the second control signal and adjust the one or more operation parameters based on the second control signal.

11. The system of claim 0, wherein the control signal is a first control signal, the system further comprising:
an observer configured to detect a sensed condition of the one or more environmental conditions over time and generate an observer signal indicating the sensed condition; and
a correction unit configured to receive the observer signal and to update the algal growth model based on the observer signal.

12. The system of claim 0, wherein the control signal is a first control signal, the system further comprising:
an observer configured to detect one or more sensed conditions of the one or more environmental conditions over time and generate an observer signal indicating the one or more sensed conditions or indicating one or more inferred conditions other than the one or more sensed conditions; and
a correction unit configured to receive the observer signal and to update the algal growth model based on the observer signal.

13. The system of claim 0, wherein the observer is configured to generate the observer signal of the one or more sensed conditions in order to minimize noise or to fill data between infrequent samples.

14. The system of claim 0, wherein the control signal is a first control signal, the system further comprising:
a sensor configured to detect a sensed condition of the one or more environmental conditions and generate a sensing signal indicating the sensed condition; and
a feedback control unit configured to receive the sensing signal, combine the sensing signal with the first control signal using a Kalman filter, and generate a second control signal based on the combination;
wherein the actuator unit is further configured to receive the second control signal and adjust the one or more operation parameters based on the second control signal.

15. The system of claim 0, wherein the photobioreactor is a flat panel photobioreactor.

16. The system of claim 0, wherein the photobioreactor is comprised of a flexible film.

17. The system of claim 0, wherein the algal growth model accounts for geometry of the photobioreactor and culture density of the algae in the media.

18. The system of claim 0, wherein the algal growth model comprises a model of water chemistry and a model of lighting.

19. The system of claim 0, wherein the algal growth model includes a look-up table, an algebraic equation, or a differential equation.

20-55. (canceled)
